(19) United States
(12) Patent Application Publication
(10) Pub. No.: US 2023/0263758 A1
(43) Pub. Date: Aug. 24, 2023

(54) COMPOSITIONS OF EDTA HAVING ACTIVITY AGAINST PLANKTONIC AND BIOFILM CELLS OF CLINICALLY RELEVANT PATHOGENS

(71) Applicant: SterileCare Inc., Markham (CA)

(72) Inventor: Karen Mueller, Newmarket (CA)

(21) Appl. No.: 18/112,674

(22) Filed: Feb. 22, 2023

Related U.S. Application Data
(60) Provisional application No. 63/312,516, filed on Feb. 22, 2022.
(60) Provisional application No. 63/312,628, filed on Feb. 22, 2022.
(60) Provisional application No. 63/396,052, filed on Aug. 8, 2022.

(51) Int. Cl. A61K 31/198; A61K 31/727; A61K 31/549; A61K 31/155; A61K 45/06

(52) U.S. Cl. CPC A61K 31/198; A61K 31/727; A61K 31/549; A61K 31/155; A61K 45/06

(57) ABSTRACT

Described herein are compositions of EDTA having activity against planktonic and biofilm cells of clinically relevant pathogens. The compositions generally include EDTA and are capable of inhibiting the growth and proliferation of microbes. The compositions may also include additional active ingredients such as heparin, thrombolytic agents, taurolidine, chlorhexidine, ethanol, and combinations thereof.

### CROSS-REFERENCE TO RELATED APPLICATIONS

This application claims priority to U.S. Provisional Application No. 63/312,516 entitled “SYNERGISTIC ACTIVITY OF TETRASODIUM EDTA, ETHANOL, AND CHLORHEXIDINE HYDROCHLORIDE AGAINST PLANKTONIC AND BIOFILM CELLS OF CLINICALLY RELEVANT PATHOGENS”, filed Feb. 22, 2022, and to U.S. Provisional Application No. 63/312,628 entitled “SYNERGISTIC ACTIVITY OF TETRASODIUM EDTA AND HEPARIN AGAINST PLANKTONIC AND BIOFILM CELLS OF CLINICALLY RELEVANT PATHOGENS”, filed Feb. 22, 2022, and to U.S. Provisional Application No. 63/396,052 entitled “MULTIPURPOSE SOLUTION FOR IMPROVED CATHETER LOCKS OR ENHANCED SAFETY OF IMPLANTABLE MEDICAL DEVICES”, filed Aug. 8, 2022, the entire contents of each of which are incorporated by reference herein.

### FIELD OF THE DISCLOSURE

The present disclosure is generally related to compositions, solutions, and methods of making, using, and testing products derived from the combination of EDTA and other active agents, wherein the products and methods may have a variety of benefits related to antimicrobial activity, biofilm formation, or preventing or eliminating blood clots, or other beneficial outcomes with respect to the use of catheters or other implantable medical devices.

### BACKGROUND

Biofilms associated with implantable medical devices and wounds are clinically relevant, often requiring repeated antibiotics without success.

The number of patients predisposed to hospital-acquired infections has been on the rise owing to an increase in patients with impaired immunity and chronic diseases and the administration of immunosuppressants or anticancer agents. Patients in the intensive care unit (ICU) are more susceptible to hospital-acquired infections than those in general wards and are susceptible to infection with pathogenic micro-organisms through various implantable medical devices. In particular, central venous access devices (CVADs) are among the most common sources of healthcare-associated bloodstream infections worldwide, with a mortality rate of 12-25%. The use of long-term CVADs is inevitable for patients admitted in nephrology, oncology, and ICUs owing to the ease of administration of blood products, fluids, parenteral nutrients, and medical therapies to the bloodstream. Unfortunately, CVADs are prone to complications such as occlusion, clot formation, and microbial colonization, all of which lead to prolonged hospitalization, expensive treatments, and significant mortality and morbidity.

Biofilms formed within CVADs are resistant to systemic antibiotic therapy alone, with 10- to 1000-fold greater resistance to conventional antibiotics than planktonic cells. Appropriate control measures and management of catheter-related infections have become a significant challenge for physicians.

To salvage long-term CVADs, the use of antimicrobial lock solutions (ALSs) has been proposed in addition to parenteral administration of antibiotics for the prevention and treatment of central line bloodstream infections (CLABSIs). Catheter lumens may be locked with highly concentrated antibiotic solutions and allowed to dwell for a specified time to fight biofilm formation. However, the prophylactic use of antibiotic locks increases concerns about the emergence of multidrug resistance among pathogens. There is clearly a need for improved lock solutions, lock flushes, catheter systems, wipes, cleaning agents, and other products for reducing infection in health care settings such as catheter use and in a variety of other settings.

### SUMMARY OF THE DISCLOSURE

Provided herein are sterile compositions that comprise a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and an additional ingredient selected from the group consisting of heparin, taurolidine, a thrombolytic agent, or a combination thereof, wherein the composition has a pH of at least 6.5 and is biocompatible in a patient's bloodstream. In some aspects, the EDTA has a concentration in the composition from about 1% (w/v) to about 10% (w/v), or from about 1% (w/v) to about 5% (w/v).

In some embodiments, the composition has a pH from about 6.5 to about 11.5, from about 6.5 to about 7.5, form about 6.5 to about 10, or from about 8.5 to about 11.

In some embodiments, the composition further comprises chlorhexidine or a pharmaceutically acceptable salt thereof. In some aspects, the chlorhexidine or a pharmaceutically acceptable salt thereof has a concentration in the composition from about 0.5% (w/v) to about 6% (w/v). In some other aspects, the chlorhexidine or a pharmaceutically acceptable salt thereof has a concentration in the composition from about 0.1 μg/mL to about 100 μg/m L.

In some embodiments, the composition further comprises ethanol. In some aspects, the ethanol has a concentration in the composition from about 0.1% (w/v) to about 70% (w/v).

In some embodiments, the additional ingredient comprises heparin, and the heparin has a concentration in the composition from about 1% (w/v) to about 8% (w/v), or from about 1% (w/v) to about 4% (w/v).

In some embodiments, the additional ingredient comprises a thrombolytic agent, and the thrombolytic agent comprises alteplase, streptokinase, reteplase, tenecteplase, urokinase, prourokinase, anistreplase, or a combination thereof. In some exemplary embodiments, the thrombolytic agent comprises alteplase, urokinase, streptokinase, or a combination thereof. In some aspects, the thrombolytic agent has a concentration in the composition of at least about 0.1% (w/v). In some additional aspects, the thrombolytic agent has a concentration in the composition from about 0.1% (w/v) to about 1.5% (w/v).

In some embodiments, the additional ingredient comprises taurolidine, and the taurolidine has a concentration in the composition from about 1% (w/v) to about 8% (w/v), or from about 1% (w/v) to about 4% (w/v).

Further provided herein are sterile compositions that comprise a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and a thrombolytic agent, wherein the composition has a pH of at least 6.5 and is biocompatible in a patient's bloodstream. In some aspects, the EDTA has a concentration in the composition from about 1% (w/v) to about 10% (w/v), or from about 1% (w/v) to about 5% (w/v).

In some embodiments, the composition has a pH from about 6.5 to about 11.5, from about 6.5 to about 7.5, form about 6.5 to about 10, or from about 8.5 to about 11.

In some embodiments, the composition further comprises chlorhexidine or a pharmaceutically acceptable salt thereof. In some aspects, the chlorhexidine or a pharmaceutically acceptable salt thereof has a concentration in the composition from about 0.5% (w/v) to about 6% (w/v). In some other aspects, the chlorhexidine or a pharmaceutically acceptable salt thereof has a concentration in the composition from about 0.1 μg/mL to about 100 μg/m L.

In some embodiments, the composition further comprises ethanol. In some aspects, the ethanol has a concentration in the composition from about 0.1% (w/v) to about 70% (w/v).

In some embodiments, the thrombolytic agent comprises alteplase, streptokinase, reteplase, tenecteplase, urokinase, prourokinase, anistreplase, or a combination thereof. In some exemplary embodiments, the thrombolytic agent comprises alteplase, urokinase, streptokinase, or a combination thereof. In some aspects, the thrombolytic agent has a concentration in the composition of at least about 0.1% (w/v). In some additional aspects, the thrombolytic agent has a concentration in the composition from about 0.1% (w/v) to about 1.5% (w/v).

Further provided herein are sterile compositions that comprise a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and taurolidine, wherein the taurolidine has a concentration of at least about 0.1% (w/v), and wherein the composition has a pH of at least 6.5 and is biocompatible in a patient's bloodstream. In some aspects, the EDTA has a concentration in the composition from about 1% (w/v) to about 10% (w/v), or from about 1% (w/v) to about 5% (w/v).

In some embodiments, the composition has a pH from about 6.5 to about 11.5, from about 6.5 to about 7.5, form about 6.5 to about 10, or from about 8.5 to about 11.

In some embodiments, the composition further comprises chlorhexidine or a pharmaceutically acceptable salt thereof. In some aspects, the chlorhexidine or a pharmaceutically acceptable salt thereof has a concentration in the composition from about 0.5% (w/v) to about 6% (w/v). In some other aspects, the chlorhexidine or a pharmaceutically acceptable salt thereof has a concentration in the composition from about 0.1 μg/mL to about 100 μg/m L.

In some embodiments, the composition further comprises ethanol. In some aspects, the ethanol has a concentration in the composition from about 0.1% (w/v) to about 70% (w/v).

In some embodiments, the taurolidine has a concentration from about 1% (w/v) to about 8% (w/v), or from about 1% (w/v) to about 4% (w/v).

Further provided herein are sterile compositions that comprise a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and heparin, wherein the taurolidine has a concentration of at least about 1% (w/v), and wherein the composition has a pH of at least 6.5 and is biocompatible in a patient's bloodstream. In some aspects, the EDTA has a concentration in the composition from about 1% (w/v) to about 10% (w/v), or from about 1% (w/v) to about 5% (w/v).

In some embodiments, the composition has a pH from about 6.5 to about 11.5, from about 6.5 to about 7.5, form about 6.5 to about 10, or from about 8.5 to about 11.

In some embodiments, the composition further comprises chlorhexidine or a pharmaceutically acceptable salt thereof. In some aspects, the chlorhexidine or a pharmaceutically acceptable salt thereof has a concentration in the composition from about 0.5% (w/v) to about 6% (w/v). In some other aspects, the chlorhexidine or a pharmaceutically acceptable salt thereof has a concentration in the composition from about 0.1 μg/mL to about 100 μg/m L.

In some embodiments, the composition further comprises ethanol. In some aspects, the ethanol has a concentration in the composition from about 0.1% (w/v) to about 70% (w/v).

In some embodiments, the heparin has a concentration from about 1% (w/v) to about 8% (w/v), or about 1% (w/v) to about 4% (w/v).

### BRIEF DESCRIPTION OF THE FIGURES

FIGS. 1A-1B illustrate an exemplary determination of the minimum biofilm eradication concentration (MBEC) for tetrasodium EDTA against gram-positive, gram-negative, and fungal biofilms. CFU/mL were enumerated from each peg (n=8) after biofilm growth for 48 h and following antimicrobial exposure for 24 h, where points on the graph represent the mean±standard deviation from three independent experiments, and statistical significance is indicated as follows: *: P<0.05; **: P<0.005; ***: P<0.0005; ****: P<0.0001.

FIGS. 2A-2B illustrate an exemplary determination of the minimum biofilm eradication concentration (MBEC) for ethanol against gram-positive, gram-negative, and fungal biofilms CFU/ml were enumerated from each peg (n=8) after biofilm growth for 48 h and following antimicrobial exposure for 24 h, where points on the graph represent the mean±standard deviation from three independent experiments, and statistical significance is indicated as follows: *: P<0.05; **: P<0.005; ***: P<0.0005; ****: P<0.0001.

FIGS. 3A-3B illustrate an exemplary determination of the minimum biofilm eradication concentration (MBEC) for chlorhexidine HCl against gram-positive, gram-negative, and fungal biofilms. CFU/mL were enumerated from each peg (n-8) after biofilm growth for 48 h and following antimicrobial exposure for 24 h, where points on the graph represent the mean±standard deviation from three independent experiments, and statistical significance is indicated as follows: *: P<0.05; **: P<0.005; ***: P<0.0005; ****: P<0.0001.

FIGS. 4A-4C illustrate an exemplary efficacy of tetrasodium EDTA (TE), ethanol (ET), and chlorhexidine HCl (CH) against gram-positive and gram-negative bacterial biofilms in a 2-h exposure period, where each column represents different concentrations of test antimicrobials against each organism tested: (i) three dark grey columns represent treatment with the MBEC of TE (%), ET (%) and CH (μg/mL); (ii) three light grey column s represents treatment with the FBEC of TE+ET, TE+CH, and TE+ET+CH; (iii) white and hatched columns represent treatment with triple combinations of TE+ET+CH, with hatched bar combinations showing the best killing effects, and statistical significance was determined by comparison with non-treated biofilms (black bar) and is indicated as follows: *: P<0.05; **: P<0.005; ***: P<0.0005; ****: P<0.0001. MBEC=minimum biofilm eradication concentration; FBEC=fractional biofilm eradication concentration.

FIG. 5 illustrates an exemplary efficacy of tetrasodium EDTA (TE), ethanol (ET), and chlorhexidine HCl (CH) against fungal biofilms in a 2-h exposure period, where each column represents different concentrations of test antimicrobials against each organism tested: (i) three dark grey columns represent treatment with the MBEC of TE (%), ET (%) and CH (μg/mL); (ii) three light grey column represents treatment with the FBEC of TE+ET, TE+CH, and TE+ET+CH; (iii) white and hatched columns represent treatment with triple combinations of TE+ET+CH, with hatched bar combinations showing the best killing effects, and statistical significance was determined by comparison with non-treated biofilms (black bar) and is indicated as follows: *: p<0.05; **: p<0.005. MBEC, minimum biofilm eradication concentration; FBEC, fractional biofilm eradication concentration.

### DETAILED DESCRIPTION

The present disclosure involves antiseptic solutions comprising, or consisting essentially of, or consisting of, one or more salt(s) of ethylene diamine tetraacetic acid (EDTA) at a prescribed concentration and/or pH. The inventors have unexpectedly discovered that such EDTA compositions provide powerful antiseptic activities and function as broad-spectrum antimicrobial agents and fungicidal agents against many strains of pathogenic yeast. EDTA combinations of the present disclosure are also highly effective in killing pathogenic biofilm organisms and in reducing and eliminating existing biofilms, as well as preventing biofilm formation.

### I. Compositions

The compositions of the present disclosure are safe for human administration and are biocompatible and non-corrosive. Preferably, the compositions are sterile. They may also have anticoagulant properties and are thus useful for preventing and/or treating a variety of catheter-related infections. The antiseptic solutions of the present disclosure have numerous applications, including applications as lock and lock flush solutions for various types of catheters, used as antiseptic agents, or solutions for sanitizing a range of medical, dental, and veterinary devices, instruments, and other objects, surfaces, and the like. They furthermore have sanitizing applications in industrial and food preparation and handling settings. The compositions may be in the form of solutions, wherein the solvent comprises water or saline.

In one embodiment, antiseptic compositions are disclosed that have at least four, and preferably at least five, of the following properties: anticoagulant properties; inhibitory and/or bactericidal activity against a broad spectrum of bacteria in a planktonic form; inhibitory and/or fungicidal activity against a spectrum of fungal pathogens; inhibitory and/or bactericidal activity against a broad spectrum of bacteria in a sessile form; inhibitory activity against protozoan infections; inhibitory activity against Acanthamoeba infections; safe and biocompatible, at least in modest volumes, in contact with a patient; safe and biocompatible, at least in modest volumes, in a patient's bloodstream; and safe and compatible with industrial objects and surfaces.

Importantly, in most embodiments, sanitizing compositions and methods of the present disclosure do not comprise traditional antibiotic agents (e.g., beta-lactams, aminoglycosides, chloramphenicol, glycopeptides, quinolones, oxazolidinones, sulfonamides, tetracyclines, macrolides, ansamycins, streptogramins, lipopeptides, etc.) and thus do not contribute to the development of antibiotic-resistant organisms.

The compositions provided herein have activity against planktonic and biofilm cells of clinically relevant pathogens. The clinically relevant pathogens generally include, bacteria, fungi, and protists. The clinically relevant pathogens include, but are not limited to, Staphylococcus (including S. epidermidis, S. aureus, and MRSA), Stenotrophomonas (including S. maltophilia), Pseudomonas (including P. Aeruginosa), Serratia (including S. marcescens), Proteus (including P. mirabilis), Escherichia (including E. coli), Klebsiella, (including K. pneumoniae), Acanthamoeba, and Candida (including C. albicans and C. parapsilosis).

The compositions provided herein comprise a salt of EDTA in solution. Sodium salts of EDTA are commonly available and may be used, including di-sodium, tri-sodium, and tetra-sodium salts, and combinations thereof. However, other EDTA salts, including ammonium, di-ammonium, potassium, di-potassium, cupric di-sodium, magnesium di-sodium, ferric sodium, and combinations thereof may also be used in addition to or instead of the sodium salts of EDTA, provided they have the antibacterial and/or fungicidal and/or anti-protozoan and/or anti-amoebic properties desired, and provided that they are sufficiently soluble in the solvent desired. In preferred embodiments, the EDTA comprises tri-sodium and tetra-sodium salts of EDTA.

The concentration of EDTA in the composition may be from about 0.5% (w/v) to about 15% (w/v), such as from about 0.5% (w/v) to about 2.5% (w/v), about 1.0% (w/v) to about 5.0% (w/v), about 1.0% (w/v) to about 7.5% (w/v), about 1.0% (w/v) to about 10% (w/v), about 1.0% (w/v) to about 12.5% (w/v), about 1.0% (w/v) to about 15% (w/v), about 2.5% (w/v) to about 15% (w/v), about 5.0% (w/v) to about 15% (w/v), about 7.5% (w/v) to about 15% (w/v), about 10% (w/v) to about 15% (w/v), or about 12.5% (w/v) to about 15% (w/v). Therefore, the concentration of EDTA in the composition may be about 1.0% (w/v), about 2.0% (w/v), about 3.0% (w/v), about 4.0% (w/v), about 5.0% (w/v), about 6.0% (w/v), about 7.0% (w/v), about 8.0% (w/v), about 9.0% (w/v), about 10% (w/v), about 11% (w/v), about 12% (w/v), about 13% (w/v), about 14% (w/v), or about 15% (w/v). Preferably, the EDTA has a concentration of at least about 1% (w/v).

In other embodiments, the EDTA may have a concentration in the composition from about 0.015% (w/v) to about 2% (w/v). For example, the EDTA may have a concentration in the composition from about 0.015% (w/v) to about 0.05% (w/v), about 0.015% (w/v) to about 0.1% (w/v), about 0.015% (w/v) to about 0.5% (w/v), about 0.015% (w/v) to about 1% (w/v), about 0.015% (w/v) to about 1.5% (w/v), about 0.015% (w/v) to about 2% (w/v), about 0.05% (w/v) to about 2% (w/v), about 0.1% (w/v) to about 2% (w/v), about 0.5% (w/v) to about 2% (w/v), about 1% (w/v) to about 2% (w/v), or about 1.5% (w/v) to about 2% (w/v). Therefore, the concentration of EDTA in the composition may be about 0.015% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.5% (w/v), or about 2% (w/v).

In other embodiments, the composition may be substantially free of EDTA (i.e., less than 0.001% (w/v)).

The composition may further comprise ethanol. The ethanol may be present at a concentration from about 0.1% (w/v) to about 70% (w/v). For example, the ethanol may have a concentration in the composition from about 0.1% (w/v) to about 1% (w/v), about 0.1% (w/v) to about 5% (w/v), about 0.1% (w/v) to about 10% (w/v), about 0.1% (w/v) to about 30% (w/v), about 0.1% (w/v) to about 50% (w/v), about 0.1% (w/v) to about 70% (w/v), about 1% (w/v) to about 70% (w/v), about 5% (w/v) to about 70% (w/v), about 10% (w/v) to about 70% (w/v), about 30% (w/v) to about 70% (w/v), about 50% (w/v) to about 70% (w/v), about 5% (w/v) to about 70% (w/v), about 10% (w/v) to about 50% (w/v), about 10% (w/v) to about 40% (w/v), about 10% (w/v) to about 30% (w/v), about 5% (w/v) to about 20% (w/v), or about 3.125% (w/v) to about 12.5% (w/v). Further, the composition may comprise ethanol in a concentration of about 0.1% (w/v), about 0.5% (w/v), about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), about 20% (w/v), about 30% (w/v), about 40% (w/v), about 50% (w/v), about 60% (w/v), or about 70% (w/v).

In some additional embodiments, the composition may comprise a weight ratio of water to ethanol from about 10:1 to about 1:10. For example, the composition may comprise a weight ratio of water to ethanol from about 10:1 to about 8:1, about 10:1 to about 6:1, about 10:1 to about 4:1, about 10:1 to about 2:1, about 10:1 to about 1:1, about 10:1 to about 1:2, about 10:1 to about 1:4, about 10:1 to about 1:6, about 10:1 to about 1:8, about 10:1 to about 1:10, about 8:1 to about 1:10, about 6:1 to about 1:10, about 4:1 to about 1:10, about 2:1 to about 1:10, about 1:1 to about 1:10, about 1:2 to about 1:10, about 1:4 to about 1:10, about 1:6 to about 1:10, or about 1:8 to about 1:10. In additional embodiments, the composition may comprise a weight ratio of water to ethanol of about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

In other embodiments, the composition may be free of ethanol or substantially free of ethanol (i.e., less than 0.001% (w/v)).

The composition may further comprise chlorhexidine or a pharmaceutically acceptable salt thereof. Other compositions and solutions derived from chlorhexidine [1,6-bis(4′-chlorophenyl biguanide) hexane] are divalent cationic biguanide agents that exist as acetate, gluconate, and hydrochloride salts. In preferred embodiments when the composition comprises chlorhexidine, the composition comprises chlorhexidine HCl. The chlorhexidine may have a concentration in the composition from about 0.5% (w/v) to about 6% (w/v), such as from about 0.5% (w/v) to about 1% (w/v), about 0.5% (w/v) to about 2% (w/v), about 0.5% (w/v) to about 3% (w/v), about 0.5% (w/v) to about 4% (w/v), about 0.5% (w/v) to about 5% (w/v), about 0.5% (w/v) to about 6% (w/v), about 1% (w/v) to about 6% (w/v), about 2% (w/v) to about 6% (w/v), about 3% (w/v) to about 6% (w/v), about 4% (w/v) to about 6% (w/v), about 5% (w/v) to about 6%, or about 1% (w/v) to about 3% (w/v).

In another embodiment, the composition may comprise chlorhexidine in a concentration from about 0.1 μg/mL to about 100 μg/mL. For example, the composition may comprise chlorhexidine in a concentration from about 0.1 μg/mL to about 0.5 μg/mL, about 0.1 μg/mL to about 1 μg/mL, about 0.1 μg/mL to about 5 μg/mL, about 0.1 μg/mL to about 10 μg/mL, 0.1 μg/mL to about 25 μg/mL, about 0.1 μg/mL to about 50 μg/mL, about 0.1 μg/mL to about 75 μg/mL, about 0.1 μg/mL to about 100 μg/mL, about 0.5 μg/mL to about 100 μg/mL, about 1 μg/mL to about 100 μg/mL, about 5 μg/mL to about 100 μg/mL, about 10 μg/mL to about 100 μg/mL, about 25 μg/mL to about 100 μg/mL, about 50 μg/mL to about 100 μg/mL, or about 75 μg/mL to about 100 μg/mL. In some additional examples, the composition may comprise chlorhexidine at a concentration of about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, or about 100 μg/mL. In an exemplary embodiment, composition may comprise chlorhexidine at a concentration from about 2.5 μg/mL to about 5 μg/mL, from about 0.4 μg/mL to about 50 μg/mL, or from about 0.1 μg/mL to about 50 μg/mL.

In other embodiments, the composition may be free of chlorhexidine or may be substantially free of chlorhexidine (i.e., less than 0.01 μg/mL).

The composition may further include taurolidine. The taurolidine may be present in the composition at a concentration from about 0.5% (w/v) to about 8% (w/v). For example, the taurolidine may be present at a concentration from about 0.5% (w/v) to about 1% (w/v), about 0.5% (w/v) to about 2% (w/v), about 0.5% (w/v) to about 3% (w/v), about 0.5% (w/v) to about 4% (w/v), about 0.5% (w/v) to about 5% (w/v), about 0.5% (w/v) to about 6% (w/v), about 0.5% (w/v) to about 7% (w/v), about 0.5% (w/v) to about 8% (w/v), about 1% (w/v) to about 8% (w/v), about 1.5% (w/v) to about 8% (w/v), about 2% (w/v) to about 8% (w/v), about 3% (w/v) to about 8% (w/v), about 4% (w/v) to about 8% (w/v), about 5% (w/v) to about 8% (w/v), about 6% (w/v) to about 8% (w/v), about 7% (w/v) to about 8% (w/v), about 2% (w/v) to about 7% (w/v), about 1% (w/v) to about 6% (w/v), or about 1% (w/v) to about 4% (w/v). The taurolidine may be present in the composition at a concentration of about 0.5% (w/v), about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), or about 8% (w/v). In other embodiments, the composition may be free of taurolidine or substantially free of taurolidine (i.e., less than 0.01% w/v taurolidine).

In other embodiments, the weight ratio of EDTA to taurolidine may range from about 0.025:1 to about 40:1. For example, the weight ratio of EDTA to taurolidine may be from about 0.025:1 to about 0.1:1, about 0.025:1 to about 0.5:1, about 0.025:1 to about 1:1, about 0.025:1 to about 2:1, about 0.025:1 to about 5:1, about 0.025:1 to about 10:1, about 0.025:1 to about 25:1, about 0.025:1 to about 40:1, about 0.1:1 to about 40:1, about 0.5:1 to about 40:1, about 1:1 to about 40:1, about 2:1 to about 40:1, about 5:1 to about 40:1, about 10:1 to about 40:1, about 25:1 to about 40:1, about 0.2:1 to about 5:1, about 0.1:1 to about 1:1, about 1:1 to about 10:1, or about 0.5:1 to about 20:1. In some additional embodiments, the composition may include heparin in a weight ratio of EDTA to taurolidine of about 0.025:1, 0.05:1, 0.075:1, 0.1:1, 0.25:1, 0.5:1, 0.75:1, 1:1, 1.5:1, 2:1, 3:1, 4:1, 5:1, 10:1, 15:1, 20:1, 25:1, 30:1, 35:1, or about 40:1. In preferred embodiments, the weight ratio of EDTA to taurolidine may range from about 0.1:1 to about 10:1, about 0.2:1 to about 5:1, about 0.1:1 to about 1:1, about 1:1 to about 10:1, or about 0.5:1 to about 20:1.

The composition may further include heparin, heparan sulfate, or a combination thereof. The combination of EDTA and heparin for catheters in contact with the bloodstream of a patient has a synergistic effect in part because of the different mechanisms of EDTA and heparin relative to catheters, and in particular due to the combination of a system anti-coagulation or anti-clotting effect that heparin can induce which can reduce the attachment of blood clots to a catheter or reduce the risk of occlusion of a catheter by blood clots. In contrast, EDTA can operate via different mechanisms to hinder the formation of biofilms on the solid surfaces of the catheter and can provide an antimicrobial effect, particularly through synergy with other agents such as chlorhexidine, taurolidine, or ethanol, against planktonic and sessile bacteria. Thus, in some aspects, the benefits of EDTA combined with heparin and optionally additional antimicrobial agents may entail synergy between local antimicrobial/antibiofilm action and systemic impact on the patient.

The composition may include heparin, heparan sulfate, or a combination thereof in a concentration of at least about 0.5% (w/v). The heparin may be present in a concentration from about 1% (w/v) to about 8% (w/v), such as from about 0.5% (w/v) to about 1% (w/v), about 0.5% (w/v) to about 2% (w/v), about 0.5% (w/v) to about 3% (w/v), about 0.5% (w/v) to about 4% (w/v), about 1% (w/v) to about 5% (w/v), about 0.5% (w/v) to about 6% (w/v), about 0.5% (w/v) to about 7% (w/v), about 0.5% (w/v) to about 8% (w/v), about 2% (w/v) to about 8% (w/v), about 3% (w/v) to about 8% (w/v), about 4% (w/v) to about 8% (w/v), about 5% (w/v) to about 8% (w/v), about 6% (w/v) to about 8% (w/v), about 7% (w/v) to about 8% (w/v), about 1.5% (w/v) to about 8% (w/v), about 2% (w/v) to about 7% (w/v). In some exemplary embodiments, the heparin may be present in a concentration from about 0.5% (w/v) to about 1.8% (w/v), about 1% (w/v) to about 2.5% (w/v), or from about 0.5% (w/v) to about 4% (w/v).

The heparin, heparan sulfate, or combination thereof may further be present in a concentration of about 0.5% (w/v), about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), or about 8% (w/v), or about 1% (w/v) to about 8% (w/v). In another embodiment, the composition may include heparin in a concentration of at least about 0.5% (w/v), at least about 1% (w/v), at least about 2% (w/v), at least about 5% (w/v), or at least about 8% (w/v). Preferably, the heparin has a concentration of at least about 1%.

In still further embodiments, the composition may be free of heparin and/or heparan sulfate or substantially free of heparin and/or heparan sulfate (i.e., less than 0.01% w/v heparin and/or heparan sulfate).

In additional embodiments, the composition may include heparin in a weight ratio of EDTA to heparin from about 0.025:1 to about 40:1. For example, the weight ratio of EDTA to heparin may be from about 0.025:1 to about 0.1:1, about 0.025:1 to about 0.5:1, about 0.025:1 to about 1:1, about 0.025:1 to about 2:1, about 0.025:1 to about 5:1, about 0.025:1 to about 10:1, about 0.025:1 to about 25:1, about 0.025:1 to about 40:1, about 0.1:1 to about 40:1, about 0.5:1 to about 40:1, about 1:1 to about 40:1, about 2:1 to about 40:1, about 5:1 to about 40:1, about 10:1 to about 40:1, about 25:1 to about 40:1, about 0.2:1 to about 5:1, about 0.1:1 to about 1:1, about 1:1 to about 10:1, or about 0.5:1 to about 20:1. In some additional embodiments, the composition may include heparin in a weight ratio of EDTA to heparin of about 0.025:1, 0.05:1, 0.075:1, 0.1:1, 0.25:1, 0.5:1, 0.75:1, 1:1, 1.5:1, 2:1, 3:1, 4:1, 5:1, 10:1, 15:1, 20:1, 25:1, 30:1, 35:1, or about 40:1.

The composition may further comprise a thrombolytic agent. Thrombolytic agents such as alteplase, urokinase, and streptokinase may be considered to deal with existing clots that cause thrombosis or occlude catheters. Such agents and their mechanisms are unrelated to EDTA's antibacterial or anti-biofilm activity and may require different conditions than those that provide optimum performance of EDTA in a catheter lock solution. Despite multiple barriers, the Applicant has examined the possibility of an unexpected synergistic effect between EDTA and thrombolytic compounds, such that novel products and methods based on combining both classes of compounds can now be provided for improved results with implantable medical devices such as catheters. Such synergistic effects may include, but are not limited to, enhanced efficacy in preventing blood clots or undermining existing clots, enhanced efficacy in biofilm mitigation or prevention, enhanced stability or lifetime of a thrombolytic agent or solution comprising a thrombolytic agent, reduced requirement for system use of thrombolytic agents in association with implantable medical devices, etc.

The thrombolytic agent may comprise a protein or protein mixture, and more particularly may comprise an enzyme or a mixture of enzymes. In some aspects, however, the thrombolytic agent does not include heparin or aspirin. In preferred embodiments, the thrombolytic agent may comprise one or more enzymes such as alteplase, streptokinase, reteplase, tenecteplase, urokinase, prourokinase, anistreplase (APSAC), etc.

“Alteplase” is a complex fibrinolytic agent, an enzyme, that is manufactured from recombinant DNA. Sometimes it is referred to as a tissue plasminogen activator (tPA). Alteplase converts plasminogen to the proteolytic enzyme plasmin, which can lyse fibrin and fibrinogen. It is often provided commercially as a lyophilized powder in, for example, 50 mg and 100 mg vials. Each vial may be packaged with diluent (e.g., sterile water for injection) for reconstitution. It is compatible with 0.9% sodium chloride (NS) and dextrose 5% water (D5W).

“Streptokinase” is an enzyme, a purified fibrinolytic bacterial protein used to break down thrombosis in situations such as myocardial infarction, pulmonary embolism, and venous thromboembolism.

“Urokinase,” also known as urokinase-type plasminogen activator (uPA), is a serine protease present in humans and other animals. It can be described as a trypsin-like enzyme that is produced endogenously by renal parenchymal cells.

“Reteplase” may also be considered. Reteplase is a recombinant tissue plasminogen activator and modified nonglycosylated form of tPA used to dissolve intracoronary emboli, promote lysis of acute pulmonary emboli, and assist the handling of myocardial infarction. Reteplase catalyzes the cleavage of endogenous plasminogen to generate plasmin. Plasmin degrades the fibrin matrix of the thrombus. Reteplase is indicated for treating acute ST-elevation myocardial infarction (STEMI) to reduce the risk of death and heart failure.

“Prourokinase” is a relatively inactive precursor that requires the conversion to urokinase to become active.

“Tenecteplase” (TNK-tPA) is a commonly used fibrinolytic agent said to be as efficient as alteplase while exerting a lower risk of non-cerebral bleeding. Tenecteplase has higher fibrin specificity and a longer plasma half-life with final clearance, mostly through hepatic metabolism.

00 37 Anistreplase is an anisoylated purified streptokinase activator complex (APSAC), a complex mixture of streptokinase and plasminogen that does not depend on circulating plasminogen to be effective.

Other known thrombolytic agents may be considered if they become approved for human or animal use. Such thrombolytic agents include, for example, Desmoteplase, a highly fibrin-specific thrombolytic experimental drug.

In preferred embodiments, the thrombolytic agent may comprise alteplase, urokinase, streptokinase, or a combination thereof.

The thrombolytic agent may be present in the composition at a concentration from about 0.01% (w/v) to about 1.5% (w/v). For example, the thrombolytic agent may be present in the composition at a concentration from about 0.01% to about 0.05% (w/v), about 0.01% (w/v) to about 0.1% (w/v), about 0.01% (w/v) to about 0.5% (w/v), about 0.01% (w/v) to about 1% (w/v), about 0.01% (w/v) to about 1.5% (w/v), about 0.05% (w/v) to about 1.5% (w/v), about 0.1% (w/v) to about 1.5% (w/v), about 0.5% (w/v) to about 1.5% (w/v), about 1% (w/v) to about 1.5% (w/v), or about 0.03% (w/v) to about 1.5% (w/v). In additional embodiments, the thrombolytic agent may be present in the composition at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), or about 1.5% (w/v). In other embodiments, the composition may be free of a thrombolytic agent, or may be substantially free of a thrombolytic agent (i.e., less than 0.001% (w/v)).

The composition may further comprise a surfactant, such as a cationic surfactant, an anionic surfactant, a zwitterionic surfactant, or combinations thereof. The surfactant may be a present at a concentration from about 0.5% (w/v) to about 20% (w/v). For example, the surfactant may be present at a concentration from about 0.1% (w/v) to about 5% (w/v), about 0.1% (w/v) to about 10% (w/v), about 0.1% (w/v) to about 15% (w/v), about 0.1% (w/v) to about 20% (w/v), about 5% (w/v) to about 20% (w/v), about 10% (w/v) to about 20% (w/v), about 15% (w/v) to about 20% (w/v). The surfactant may be present at a concentration of about 0.1% (w/v), about 0.5% (w/v), about 1% (w/v) about 1.5% (w/v), about 2% (w/v), about 2.5% (w/v), about 3% (w/v), about 4% (w/v), about 5% (w/v), about 6% (w/v), about 7% (w/v), about 8% (w/v), about 9% (w/v), about 10% (w/v), about 11% (w/v), about 12% (w/v), about 13% (w/v), about 14% (w/v), about 15% (w/v), about 16% (w/v), about 17% (w/v), about 18% (w/v), about 19% (w/v), or about 20% (w/v). In exemplary embodiments, the surfactant may be present at a concentration from about 0.5% (w/v) to about 20% (w/v), about 0.5% (w/v) to about 5% (w/v), about 0.1% (w/v) to about 1.5% (w/v), or less than about 2% (w/v).

The composition of the present disclosure may have a pH from about 6.5 to about 11.5. For example, the composition may have a pH from about 6.5 to about 7.5, about 6.5 to about 8.5, about 6.5 to about 9.5, about 6.5 to about 10.5, about 6.5 to about 11.5, about 7.5 to about 11.5, about 8.5 to about 11.5, about 9.5 to about 11.5, about 10.5 to about 11.5, about 6.5 to about 8, about 9.5 to about 11.5, about 9 to about 11, or about 7 to about 10. The composition may have a pH of about 6.5, 7.0, 7.5, 8.0, 8.5, 9.0, 9.5, 10.0, 10.5, 11.0, or about 11.5. In some exemplary embodiments, the composition may have a pH higher than physiological pH.

In one version, the composition may be initially at a first pH relatively closer to physiological pH, and then the pH may be increased to a pH of 8.5 or higher. Without wishing to be bound by theory, it is believed that the use of two distinct pH ranges can, in some aspects, allow a thrombolytic agent to be effective over a time period sufficiently long to act effectively against clots while at a pH relatively closer to physiological pH, while the EDTA can be more effective in its antimicrobial and/or anti-biofilm functions at the higher pH range, thereby allowing both compounds to have relatively optimum performance.

Thus in an exemplary aspect, a multipurpose solution is disclosed comprising at least one salt of ethylene diamine tetraacetic acid (EDTA) in solution, wherein at least one EDTA salt is tri-sodium or tetra-sodium EDTA, at a combined tri-sodium and tetra-sodium EDTA concentration of at least 1.0% (w/v) and less than 15% (w/v), further comprising at least 0.01% of a thrombolytic agent such as from 0.03% to 1% by weight of at least one of alteplase, urokinase, and streptokinase, wherein the multipurpose solution is obtained by combining the first solution at a first pH with a second solution at a second pH. In some aspects, the first solution comprises at least 0.03% of a thrombolytic agent, and the second solution comprises one or more salts of EDTA at a second pH. In some aspects, combining comprises the removal of a portion of the first solution followed by the addition of the second solution. In some aspects, the act of combining comprises the mixing of the first and second solutions. In some aspects, a pH control agent is added to the multipurpose solution after the first and second solutions are combined.

The composition may further comprise a buffering agent to control the pH. Suitable buffering agents for use in the composition are generally well known in the art, and may include citrate buffers, acetate buffers, and phosphate buffers.

The composition may demonstrate a broad-spectrum antimicrobial activity on a variety of planktonic and biofilm cells of clinically relevant pathogens and of sessile cells. The compositions may also eliminate a 48 hour old biofilm after a 2-hour exposure and provide a substantial reduction in biofilm cells within a 2-hour contact time.

In general, the composition is capable of eliminating greater than or equal to 75% of the strains of planktonic cells. In various embodiments, the composition may eliminate greater than or equal to 75%, greater than or equal to 80%, greater than or equal to 85%, greater than or equal to 90%, greater than or equal to 95%, or greater than or equal to 99% of the strains of planktonic cells.

Generally, the composition may eliminate greater than or equal to 75% of the strains of biofilm cells. In various embodiments, the composition eliminates greater than or equal to 75%, greater than or equal to 80%, greater than or equal top 85%, greater than or equal to 90%, greater than or equal to 95%, or greater than or equal to 99% of the strains of biofilm cells.

Preferably, the composition eliminates more than 95% of the planktonic cells. In various embodiments, the composition eliminates more than 95%, more than 96%, more than 97%, more than 98%, or more than 99% of the planktonic cells.

Preferably, the composition eliminates more than 95% of the biofilm cells. In various embodiments, the composition eliminated more than 95%, more than 96%, more than 97%, more than 98%, or more than 99% of the biofilm cells.

Preferably, the composition eliminates more than 95% of the sessile cells. In various embodiments, the composition eliminates more than 95%, more than 96%, more than 97%, more than 98%, or more than 99% of the sessile cells.

The compositions may eliminate greater than or equal to 99% biofilms following 24 hours of treatment or exposure.

### II. Applications

Several illustrative applications for the compositions described herein are now set forth. In such applications involving two or more different versions of the solutions described herein, it is understood that any two such different types of solutions may comprise similar ingredients, but at different concentrations, or ingredients that are not found in another type of solution. For example, a first composition may have a component such as EDTA, taurolidine, chlorhexidine, or ethanol whose concentration is at least 20% higher, 50% higher, or 100% higher (alternatively, at least 15% lower, 30% lower, or 50% lower) than the concentration of the same component in a second composition, or on an absolute weight percentage basis may have a concentration at least 0.5%, 1%, 2%, 5% or 8% greater (alternatively, lower by at least 0.3%, 0.5%, 1%, 2%, or 10%) than the concentration of the corresponding component in the second composition, such as 0.5% taurolidine in one solution and 1.5% taurolidine in the other, for an absolute difference of 1 wt %. Also, by way of example, the first composition may have 2% taurolidine while the second composition is substantially free of taurolidine, or the first composition may have from 10% to 30% ethanol, while the second composition may have 40% or more ethanol. Because the purpose of the first composition may be primarily to prevent occlusion of a catheter or prevent biofilm formation on or in a catheter, the first composition may be adapted to be biocompatible with the patient's physiology, while the second composition in some aspects need not be. The first composition and the second composition may be any of the compositions described in Section I above.

Reasons for having more than one composition provided in many aspects described herein include (1) the variability in microbes over time, wherein the composition of microbes affecting the catheter or the patient may shift in terms of the distribution and prevalence of species, requiring different antimicrobial strategies over time; (2) the changes over time that occur in a biofilm as once planktonic bacteria become sessile as they attach to a solid surface to form a biofilm, and as the biofilm matures through different stages, wherein different strategies (e.g., differing concentrations of ingredients or different ingredients) may be needed to prevent blockage of a catheter or reduce the risk of infection; (3) changes in the physiology of a patient; and (4) differences in tasks that need to be performed, such as disinfecting hands versus preparing a catheter site, or versus flushing a catheter or providing catheter lock solution.

For any given task such as flushing a catheter, more than one type of composition may also be provided, such as two or more flush solutions, two or more lock solutions, two or more skin cleansing solutions, etc., in order to cope with ongoing changes in bacteria, biofilm stage, patient physiology, and condition, etc. Such multiple versions of solutions may have varying antimicrobial concentrations or other ingredients to provide options to cope with changes over time or for different problems encountered.

Providing two or more pH values within a single implantable medical devices such as a catheter can be achieved in a variety of ways, including: (1) first filling a catheter line with a first solution comprising a thrombolytic agent at a suitable first pH, then displacing a portion of the first solution with a second solution of an EDTA salt or salts at a second pH, wherein the act of displacing may comprise flushing out some of the first solution or withdrawing it from a line that it is in as the second solution flows in to replace the removed portion of the first solution; (2) first instilling a catheter line with a first solution comprising an EDTA salt or salts at a first pH, then displacing a portion of the first solution with a second solution comprising a thrombolytic agent at a second pH, wherein the act of displacing may comprise flushing out some of the first solution or withdrawing it from a line that it is in as the second solution flows in to replace the removed portion of the first solution; (3) instilling a solution at a first pH into a catheter line, wherein the catheter line comprises a coating with a water soluble pH control agent, such that over a period of time, the solution transitions to a second pH as the pH control agent dissolves into the multipurpose solution, thereby altering its pH. The first pH may be a pH near physiological pH, and the coating on the lumen of the catheter line may comprise an alkaline material that may comprise sodium hydroxide or other alkaline agents. The solutions may be any of the compositions described in Section I above.

Time release technologies may be used to control the timing of the pH change to ensure an adequate time of exposure of the first pH to the catheter and then adequate time for the second pH to be active in the catheter line. The first pH may be near the physiological pH, and the second pH may be relatively elevated, or vice versa.

In one embodiment, the compositions disclosed herein comprising one or more sodium salt(s) of EDTA at a pH higher than physiological pH are provided as antiseptic compositions of the present invention. Such antiseptic compositions have applications as lock solutions and lock flush solutions for various types of in-dwelling access catheters, including vascular catheters used for delivery of fluids, blood products, drugs, nutrition, withdrawal of fluids or blood, dialysis, and monitoring of patient conditions, and the like. Antiseptic solutions of the present invention may also be used as lock and lock flush solutions for urinary catheters, nasal tubes, throat tubes, and the like. In one embodiment, an antiseptic solution consisting of, consisting essentially of, or comprising one or more sodium EDTA salt(s) at a pH higher than physiological pH is provided to maintain the patency of in-dwelling intravascular access devices. Methods for sanitizing catheters and other medical tubes, such as nasal tubes, throat tubes, and the like, are also provided and involve contacting the catheter or other medical tube with a sanitizing composition of the present invention.

In another embodiment, antiseptic compositions disclosed herein comprising one or more sodium salt(s) of EDTA at a pH greater than physiological pH are provided as sanitizing solutions for medical devices such as dentures and other dental and/or orthodontic and/or periodontal devices, for contact lenses and other optical devices, for medical and veterinary instruments, devices, and the like, and as sanitizing solutions for sanitizing surfaces and objects. Methods of sanitizing such devices are also provided, the methods comprising contacting a device with antiseptic compositions of the present invention. In general, antiseptic compositions of the present invention may be used as soaking solutions for dental, orthodontic, and periodontal devices, including toothbrushes, and are also used as soaking solutions for contact lenses and other optical devices, as well as medical and veterinary instruments, devices, and the like. For these applications, antiseptic compositions of the present invention are generally formulated as solutions or provided in a dry form which, upon introducing a suitable solvent, forms a solution.

In yet another embodiment, the compositions disclosed herein may be formulated for solutions, gels, creams, and other preparations designed for topical use as antiseptic agents, wipes, antibacterial treatments, and the like. Antiseptic compositions of the present invention may also be used as antibacterial agents in connection with bandages, dressings, wound healing agents and devices, sprays, and the like.

In still another embodiment, the compositions of the present disclosure may be used in industrial settings such as water storage and distribution systems, water purification, humidification, and dehumidification devices, and in food preparation, handling, and packaging settings to inhibit, reduce or substantially eliminate microbial populations in both planktonic and sessile forms, as well as many fungal, amoebic and planktonic populations. Industrial equipment and surfaces may be contacted or flushed with or soaked in antiseptic compositions of the present invention. Time-release antiseptic composition formulations may also be provided to provide treatment over time, particularly in locations that are difficult to access frequently.

Products comprising taurolidine and EDTA according to the various aspects described herein may be provided in the form of catheter lock solutions suitable for use in a wide variety of catheters, including indwelling catheters and short-term catheters and various catheters for venous and arterial access such as peripheral catheters, midline catheters, tunneled and non-tunneled central venous catheters, pulmonary artery catheters, implantable catheters, umbilical catheters, and the like. The solutions and compositions disclosed herein may also be adapted for use in various products for use with catheters and catheterization, such as wipes, caps for disinfecting catheter hubs or other devices, disinfectant or preparatory sprays or solutions, etc., or may be adapted for a wide variety of other medical and cleaning products such as scrubs, sprays, or wipes for disinfecting skin or hands, wound treatments, solid surface disinfectants, and cleaners, etc. The compositions may be provided in many formats, such as in wipes, solutions in bottles, or other dispensing means, and as part of a kit comprising various swabs, wipes, sprays, solutions, and other items to aid in cleaning or disinfecting or catheter preparation and maintenance, etc. The solutions may also be combined with soaps or surfactants to provide cleaning materials, ointments, etc. They may be provided as a coating or other treatment applied to solid surfaces, including being embedded on a surface or provided with encapsulation for controlled release from a surface or other material, such as being applied to or combined with materials used in a catheter such as on or in tubing, hubs, caps, etc.

A catheter kit may comprise two or more variations of any of the compositions described herein. For example, the kit may comprise a catheter lock solution, a flush solution, two or more kinds of wet wipes comprising two or more kinds of solution, a swab for treating sites on the catheter or catheter hub, a hand cleanser solution, and a surgical site prep solution for sterilizing a catheter site prior to inserting a catheter. The lock solution may be any one of the compositions described in Section I above. By way of example, the lock solution may comprise from 1% (w/v) to 15% (w/v) EDTA such as from 1% (w/v) to 10% (w/v) EDTA, 0.5% (w/v) to 3% (w/v) heparin such as from 0.5% (w/v) to 1.5% (w/v) heparin, and the balance of the solution aqueous saline solution (e.g., 0.9% saline). In other versions, the lock solution may also comprise from 5% (w/v) to 70% (w/v) ethanol, such as 10% (w/v) to 50% (w/v), 10% (w/v) to 40% (w/v), or 10% (w/v) to 30% (w/v) ethanol. In related examples, any of the aforementioned lock solution versions may also comprise chlorhexidine, such as from 0.5% (w/v) to 4% (w/v) chlorhexidine or from 0.5% (w/v) to 2.5% (w/v) chlorhexidine.

The flush solution may be any composition provided in Section I above. The flush solution may comprise a lower concentration of ethanol, a lower concentration of EDTA, and a higher heparin concentration as compared to the lock solution. By way of example, wet wipe solutions and solutions for swabs may comprise high levels of ethanol and be relatively low in EDTA but high in chlorhexidine and/or heparin, such as having 40% to 70% ethanol, 1% to 3% EDTA, 1% to 4% chlorhexidine, and 1.5% to 3% heparin. Hand and skin cleansers may have similar compositions to wet wipes but with variations in antimicrobial ingredients, such as higher concentrations of one or more ingredients or two or more ingredients. By way of further example, the pH of any of the solutions may range from 6.5 to 11.5, such as from 6.5 to 8, from 9.5 to 11.5, from 9 to 11, from 7 to 10, etc.

Such a kit may comprise two or more types of lock solution, such as a first lock solution or a first flush solution with a heparin concentration at least 30% higher than that of a second lock solution or second flush solution, respectively.

Another catheter kit provided herein comprises a plurality of vials comprising a predetermined quantity of a powder comprising EDTA, one or more thrombolytic agents such as alteplase or other agents, and a buffering agent is further provided herein. Other agents may be present, such as a citrate salt (e.g., sodium or potassium citrate), sodium chloride, and other salts. Catheter lock solution can be prepared by reconstituting the powder in the vial by injecting or adding a predetermined quantity of saline, water, or other suitable solution into the vial. After shaking or waiting a period of time to ensure that the powder has completely dissolved to form a multipurpose solution, the multipurpose solution can then be withdrawn by a syringe or other means to inject the multipurpose solution into a catheter line to serve as a catheter lock. The pH of the solution may be about 9 or greater, such as from 9 to 11, such that the EDTA is largely in the form of tetrasodium EDTA and/or trisodium EDTA for enhanced antimicrobial and anti-biofilm efficacy while also being suitable for the function of the thrombolytic agent, such that bacterial biofilm is prevented from forming and blood clots are much less likely to form.

In addition to the vials that may be reconstituted to form a catheter lock solution, the catheter kit may further comprise containers of solutions or powders for forming one or more solutions by reconstitution such that the solutions may serve as a flush solution, a solution for cleaning solid surfaces with wipes or swabs, a hand cleanser solution, and a surgical site prep solution for sterilizing a catheter site prior to inserting a catheter. One or more of these aforementioned solutions may also comprise chlorhexidine or other antimicrobials, such as from 0.5% (w/v) to 4% (w/v) chlorhexidine or from 0.5% (w/v) to 2.5% (w/v) chlorhexidine. The pH may of any of the solutions may range from 6.5 to 11.5, such as from 6.5 to 8, from 9.5 to 11.5, from 9 to 11, from 7 to 10, etc.

For enhanced biocompatibility or enhanced prevention of thrombosis, the catheter tubing and other associated material may be coated with suitable materials such as synthetic biocompatible polymer material comprising the reaction product of at least one polar non-ionic macromer component; at least one anionic component; and at least one hydrophobic component. Alternatively, the catheter line may be a fluoropolymer-immobilized, liquid perfluorocarbon-coated central catheter line.

A catheter lock solution described herein comprises from 1% (w/v) to 15% (w/v) EDTA such as from 1% (w/v) to 10% (w/v) EDTA, 0.01% (w/v) to 3% (w/v) thrombolytic agent such as from 0.03% (w/v) to 1.5% (w/v) or from 0.05% (w/v) to 1% (w/v), or from 0.05% (w/v) to 0.5% (w/v) thrombolytic agent, and the balance of the solution aqueous saline solution (e.g., 0.9% saline). The catheter lock solution is a multipurpose solution effective in reducing clot formation or mitigating clots and biofilm formation or the risks of bacterial infection. In other embodiments, the lock solution may also comprise 0.1% (w/v) to 5% (w/v) of a citrate salt (e.g., sodium or potassium citrate). The lock solution may also comprise 5% to 70% ethanol, such as from 10% (w/v) to 50% (w/v), 10% (w/v) to 40% (w/v), or 10% (w/v) to 30% (w/v) ethanol. In related embodiments, any aforementioned solutions may also comprise chlorhexidine, such as from 0.5% (w/v) to 4% (w/v) chlorhexidine or from 0.5% (w/v) to 2.5% (w/v) chlorhexidine.

The lock solution may be provided in pre-filled syringes that can be used to directly fill a catheter line. Alternatively, the lock solution may be provided in vials adapted for removal of the lock solution using a syringe. In some aspects, the lock solution may be in a bottle or other containers that can be used to fill multiple vials or that permits filling multiple syringes. Any other useful system for providing the lock solution and instilling it into catheter lines or into other systems for the maintenance or preparation of implantable medical devices may be considered.

In related aspects, the lock solution may be one of a plurality of lock solutions provided in a kit for catheter use and maintenance. The kit may comprise two or more distinct types of multipurpose solutions.

A wound care product may comprise any of the compositions described in Section I above. In particular, the potential benefit is considered of heparin in the care of certain types of wounds, such as described in L. Galvan. “Effects of heparin on wound healing,” Journal of Wound, Ostomy, and Continence Nursing, 23, no, 4 (July 1996): 224-6, doi: 10.1016/s1071-5754(96)90095-9, https://pubmed.ncbi.nlm.nih.gov/8900676/. The use of the related compound heparan sulfate in combination with or instead of heparin may be considered. See also P. Olczyk, et al., “Diverse Roles of Heparan Sulfate and Heparin in Wound Repair,” BioMed research international (2015): 549417, https://doi.org/10.1155/2015/549417, and https://www.ncbi.nlm.nih.gov/pmc/articles/PMC4508384/.

The wound care product may include a wrap, medicated bandage, medicated gauze pad, a spray, an ointment, a swab, etc., that can be placed in contact with a wound or surgical site such as a catheter site to reduce the risk of infection. The concentration of water or ethanol may be relatively low in the product, with highly concentrated antimicrobials provided in a thin layer of cream. Such a cream or ointment may have a carrier similar to commercial antibiotic ointments comprising lipids and other ingredients, such as cocoa butter, cottonseed oil, olive oil, sodium pyruvate, tocopheryl acetate, and white petrolatum. In some examples, the pH may range from 6.5 to 10, such as from 6.5 to 8.

A solid-surface treatment may be in the form of a spray sprayed by a manual sprayer or aerosol can or applied by a wipe or scrub that is either prewetted or is wetted immediately before use. Any of the compositions described in Section I may be suitable for use in the solid-surface treatment. The solid surface treatment may be adapted to attack or prevent biofilms that form on a variety of solid surfaces in health care settings, home settings, and other settings such as school lavatories, food preparation sites, etc., including on tools, devices, fabrics, patches, and other materials that may make temporary contact with a wound site, a surgical incision or opening, a bloodstream, a catheter, etc. Two or more types of compositions may be provided in a variety of formats, such as a wet wipe, a spray, solid or liquid soap, etc. By way of example, a first type of solid surface disinfectant may comprise a carrier of water and ethanol in a ratio by weight such as from 1:10 to 1:3, 1:5 to 1:1, 3:1 to 6:1, etc. In some examples, the EDTA concentration may range from 0.5% (w/v) to 11% (w/v), such as from 0.5% (w/v) to 5% (w/v), or some versions of the solid-surface treatment may be substantially free of EDTA. Optionally, heparin may range from 0.5% (w/v) to 4% (w/v) such as from 0.5% (w/v) to 1.8% (w/v) or from 1% (w/v) to 2.5% (w/v). Optionally, chlorhexidine may range from 0.5% (w/v) to 6% (w/v), such as from 0.5% (w/v) to 2% (w/v) or from 1% (w/v) to 3% (w/v), or some versions of the solid-surface treatment may be substantially free of chlorhexidine. In some cases, a surfactant such as anionic, cationic, or zwitterionic surfactants may also be present at a concentration of from 0.5% (w/v) to 20% (w/v), 0.5% (w/v) to 5% (w/v), from 0.1% (w/v) to 1.5% (w/v), or less than 2% (w/v).

The compositions described herein may be adapted for controlled release in various products, wherein heparin or the antimicrobial ingredients are provided, for example, in capsule form by microencapsulation such as the technologies currently used by Encapsys (Appleton, Wis.), as described, for example, in U.S. Pat. Nos. 11,180,714, 10,894,908, 10,456,766, 10,920,177, and US Patent Application 20200122110. Such microcapsules could be provided on the interior or exterior walls of a catheter, on clothing, on frequently touched solid surfaces, in wound dressings or wraps, attached to the surface of a portion of a catheter or surgical implant, or device, etc. Release of the active agents can be achieved by the dissolution of the capsule walls in a fluidic environment, by breakage of capsules during use, or diffusion through the capsule wall. Alternatively, heparin and/or EDTA may be embedded in portions of the solid materials of a catheter, such as within the walls of a silicone catheter or catheters comprising other solid materials. Swelling of the silicone or other substrate may be done in a first environment allowing the material to swell and have increased micropores, at which time heparin or EDTA may diffuse effectively in the substrate, only to be slowly but effectively released when in a bloodstream, a volume of urine, or other fluidic environments. Similar strategies may be used to pretreat an implantable or insertable object such that heparin and/or EDTA and optionally other agents such as taurolidine may be gradually released to help prevent biofilm formation in various settings, such as bone implants, dental implants, pacemakers, artificial heart valves, etc. Such strategies can be supplemented with systemic treatments provided by other means.

In another aspect, a solution of a thrombolytic agent is provided comprising EDTA, wherein the solution of the thrombolytic agent has improved shelf life and/or improved thermal stability relative to a similar solution without the EDTA. In one aspect, the solution is provided by combining the thrombolytic agent in either powder form or solution form with the EDTA in either solution form or powder form. In another aspect, a multipurpose solution is provided by reconstituting a solid or concentrated combination of EDTA and the thrombolytic agent, wherein the solid or concentrated combination has enhanced thermal stability or shelf life relative to the thrombolytic agent without the EDTA in a similar form. In one aspect, the multipurpose solution is prepared by preparing a solution from a powder form comprising both EDTA and the thrombolytic agent.

A catheter is further provided with a coated internal surface adapted to provide a pH control agent when in contact with a catheter lock solution such that the catheter lock solution experiences a change in pH over time. The coating may comprise agents that have diffused into the tubing material, such as a silicone or polyurethane tube, or may be largely provided on the internal surface of the catheter line, held in place with a soluble film or capsule walls. Microcapsules comprising the pH control agent may be attached to the catheter tube surface by adhesives or other means.

In some aspects, the catheter is provided with a porous layer on its inner surface into which various agents can be infused or which can be impregnated with agents such as pH control agents. In contact with a multipurpose solution, the pH control agent in the pores of the porous surface can dissolve and enter the multipurpose solution to cause the desired change in its pH.

The catheter can be prepared by injecting a quantity of a multipurpose solution comprising both EDTA salts and a thrombolytic agent such as alteplase, urokinase, other known thrombolytic agents, or any combination thereof. The multipurpose solution is initially at a first pH most suitable for one of the thrombolytic agents and the salts of EDTA. For example, the multipurpose solution may initially have a pH of about 6, 6.5, 7, or 7.3, such as from 6 to 8, from 6.5 to 8, or from 7.5 to 8.5. After being instilled into the catheter tube, an alkaline material embedded on or within the catheter line dissolves into the catheter lock solution, resulting in a pH that rises to at least about 8, 8.5, 9, or 9.5, such as from 8 to 11, 8.5 to 11, 9 to 11, etc.

Alternatively, the initial pH of the multipurpose solution may be significantly higher than the physiological pH, such as from 9 to 11, 9.5 to 11, 9.5 to 10.5, or 10 to 11, such that the EDTA salts in the multipurpose solution are largely in the tetrasodium or trisodium form. The associated catheter tube has a coating in the lumen or material embedded within the lumen wall. An acidic agent such as citric acid (or other pharmacologically acceptable acids) is released into the multipurpose solution when the multipurpose solution is in contact with the catheter line. After a predetermined period of time, such as about 5 minutes or more, 10 minutes or more, or 15 minutes or more, the pH of the multipurpose solution may have dropped significantly to, for example, 6.5 to 9.5, 6.5 to 9, or from 6 to 8.5, etc., reaching a level where the thrombolytic agent is particularly effective.

### III. Methods

Methods for inhibiting the growth and proliferation of microbial populations and/or fungal pathogens, including inhibiting the formation and proliferation of biofilms, are provided herein. The methods comprise contacting an infected or suspected infected object, or surface, with a composition of the present disclosure. Any composition described in Section I above may be used. Methods for inhibiting the growth and proliferation of protozoan populations are provided. The methods comprise contacting an infected or suspected infected object, or surface, with a composition of the present disclosure. Any composition described in Section I above may be used. Methods for inhibiting the growth and proliferation of amoebic populations and preventing amoebic infection, particularly Acanthamoeba infections, are provided. The methods generally comprise contacting an object, or a surface, with a composition of the present disclosure. Any composition described in Section I above may be used.

Methods for substantially eradicating microbial populations, including both planktonic microbial populations and microbial populations in the form of biofilms, are also provided. The methods comprise contacting an infected or suspected infected object, or surface, with a composition of the present disclosure. Any composition described in Section I above may be used.

In another aspect, the method of treating an implantable medical device is provided comprising contacting a portion of the implantable medical device such as a catheter with a combination of an effective amount of a thrombolytic agent and an effective amount of one or more salts of EDTA. In a related aspect, the method may include the step of contacting the implantable medical device with the first solution at a first pH, followed by the addition of a second solution at a second pH, which yields a multipurpose solution having a pH intermediate to the first and second pH.

In another aspect, a method of preparing a catheter line for a patient is provided wherein the catheter line is provided with a lock solution that is a multipurpose solution with components and concentrations adapted for the patient's individual needs. An automated system such as a software program or app is provided that considers data regarding the individual patient's health and risk factors, including the potential for blood clot formation, the risk of infection in light of the patient's immune state, age, and health, etc. Thus, for example, a patient with a history of thrombosis or adverse reactions to catheters may be provided with a lock solution with an elevated level of one or more thrombolytic agents in combination with one or more EDTA salts. In contrast, a patient with reduced risk of clot formation may be provided with a catheter lock solution with a substantially lower concentration of the thrombolytic agent. Any composition described in Section I above may be used as the catheter lock solution.

Depending on the composition used in the various methods, various compositions and contact time periods may be required to inhibit the formation and proliferation of various populations and/or to substantially eradicate various populations. Suitable contact time periods for various compositions are provided in the examples and may be determined by those having ordinary skill in the art.

The present disclosure further encompasses methods for preparing the compositions of the present disclosure. The method comprises: (a) contacting CH powder and water forming a mixture; (b) heating the mixture to about 45° C. to about 55° C. forming a chlorhexidine HCl solution; (c) cooling the chlorhexidine HCl solution to room temperature and passing the solution through a filter; and (d) contacting the chlorhexidine HCl solution, a solution of EDTA, ethanol, and optionally a solution comprising heparin, optionally a solution comprising a thrombolytic agent, and optionally a solution comprising taurolidine, thereby forming the composition.

The methods may be conducted in a batch, semi-continuous, or continuous fashion. The methods may also be conducted under an inert atmosphere such as nitrogen, helium, argon, or a combination thereof.

The method commences by contacting chlorhexidine HCl powder and water forming a mixture. Generally, the chlorhexidine HCl powder and water used in the mixture may be added in any sequential order, in portions, or all at same time.

The water used in the method may be purified, distilled, doubly distilled, or deionized water, or water for injection.

Various forms of mixing may be utilized in the method. Non-limiting examples of mixing may be magnetic mixing or mechanical mixing.

The next step in the method comprises heating the mixture to about 45° C. to about 55° C. forming a chlorhexidine HCl solution of about 1.0 mg/mL. This step utilizes the appropriate mixer as used in step (a) to ensure a solution is prepared.

The temperature of heating the chlorhexidine HCl powder and water from step (a) may range from about 45° C. to about 55° C. In various embodiments, the temperature of heating the chlorhexidine HCl powder and water may range from about 45° C. to 55° C., from 45° C. to about 48° C., from about 48° C. to about 50° C., from about 50° C. to about 53° C., or from about 53° C. to about 55° C.

In general, the duration of heating the mixture from step (b) may range from about 30 seconds to about 30 minutes until a homogeneous solution is seen visually. In various embodiments, the duration of heating the mixture from step (a) may range from about 30 seconds to about 30, from about 1 minute to about 15 minutes, or from about 15 minutes to about 30 minutes.

(c) cooling the solution to room temperature and passing the solution through a micron filter

The next step in the method comprises cooling the solution to room temperature and passing the solution through a micron filter. The micron filter may be 0.22 μm filter, a 0.20 μm filter, or a 0.10 μm filter. One or more-micron filters may be used in step (c). In one embodiment, the micron filter may be a 0.22 μm filter.

This method step removes undissolved material by passing the room temperature solution through a micron filter. The filter may be an inline micron filer, a sparkler, or a standalone filter apparatus.

The last step in the method comprises (d) contacting the chlorhexidine HCl solution, a solution of EDTA, ethanol, and optionally a solution comprising heparin, optionally a solution comprising a thrombolytic agent, and optionally a solution comprising taurolidine, thereby forming the composition. In general, the components of the composition may be added in any sequential order, in portions, or all at same time.

Various forms of mixing may be utilized in the method. Non-limiting examples of mixing may be magnetic mixing or mechanical mixing.

The temperature of contacting the components of the composition in step (d) may range from about 10° C. to about 40° C. In various embodiments, the temperature contacting the components of the composition in step (d) may range from about 10° C. to 40° C., from 15° C. to about 35° C., from about 20° C. to about 30° C. In one embodiment, the temperature of contacting the components of the composition in step (d) may be about 23° C. (room temperature).

### Definitions

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms have been provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the terms “a,” “an,” and “the” are understood to encompass the plural as well as the singular. Thus, the term “a mixture thereof” also relates to “mixtures thereof” and the term “a component” also refers to “components.”

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/m L” should also be understood to provide support for the range of “50 mg/m L to 80 mg/m L.”

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open-ended term, like “comprising” or “including,” it is understood that direct support should be afforded also to “consisting essentially of” language as well as “consisting of” language as if stated explicitly and vice versa.

As used herein, “implantable medical devices” may include catheters and other medical implants to promote wound healing or other aspects of health. Compositions according to the various aspects described herein, may be provided in the form of catheter lock solutions suitable for use in catheters as the implantable medical device and may be considered for a wide variety of catheters, including indwelling catheters and short-term catheters and various catheters for venous and arterial access such as peripheral catheters, midline catheters, tunneled and nontunneled central venous catheters, pulmonary artery catheters, implantable catheters, umbilical catheters, and the like. The solutions and compositions disclosed herein may also be adapted for use in various products for use with catheters and catheterization, such as wipes, caps for disinfecting catheter hubs or other devices, disinfectant or preparatory sprays or solutions, etc., or may be adapted for a wide variety of other medical and cleaning products such as scrubs, sprays, or wipes for disinfecting skin or hands, wound treatments, solid surface disinfectants, and cleaners, etc. The compositions may be provided in many formats, such as in wipes, solutions in bottles, or other dispensing means, and as part of a kit comprising various swabs, wipes, sprays, solutions, and other items to aid in cleaning or disinfecting or catheter preparation and maintenance, etc. The solutions may also be combined with soaps or surfactants to provide cleaning materials, ointments, etc. They may be provided as a coating or other treatment applied to solid surfaces, including being embedded on a surface or provided with encapsulation for controlled release from a surface or other material, such as being applied to or combined with materials used in a catheter such as on or in tubing, hubs, caps, etc.

As used herein, “long-term” with respect to the use of a catheter or other implantable medical device can refer to a time of at least 8 hours, 12 hours, 18 hours, 24 hours, 32 hours, or at least an integral number of days from 2 to 120. A range of time for a “long-term” period can extend from any of the above-mentioned time periods to a time of any integral number of days.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

### EXEMPLARY EMBODIMENTS

Embodiment 1: A sterile composition comprising:
a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and
an additional ingredient selected from the group consisting of heparin, taurolidine, a thrombolytic agent, or a combination thereof,
wherein the composition has a pH of at least 6.5 and is biocompatible in a patient's bloodstream.

Embodiment 2: The composition of embodiment 1, wherein the composition has a pH from about 6.5 to about 11.5.

Embodiment 3: The composition of embodiment 2, wherein the composition has a pH from about 6.5 to about 7.5.

Embodiment 4: The composition of embodiment 2, wherein the composition has a pH from about 6.5 to about 10.

Embodiment 5: The composition of embodiment 2, wherein the composition has a pH from about 8.5 to about 11.

Embodiment 6: The composition of any one of embodiments 1-5, further comprising chlorhexidine or a pharmaceutically acceptable salt thereof.

Embodiment 7: The composition of embodiment 6, wherein the chlorhexidine or a pharmaceutically acceptable salt thereof has a concentration in the composition from about 0.5% (w/v) to about 6% (w/v).

Embodiment 8: The composition of embodiment 6, wherein the chlorhexidine or a pharmaceutically acceptable salt thereof has a concentration in the composition from about 0.1 μg/mL to about 100 μg/m L.

Embodiment 9: The composition of any one of embodiments 1-8, further comprising ethanol.

Embodiment 10: The composition of embodiment 9, wherein the ethanol has a concentration in the composition from about 0.1% (w/v) to about 70% (w/v).

Embodiment 11: The composition of any one of embodiments 1-10, wherein the additional ingredient comprises heparin, and the heparin has a concentration in the composition from about 1% (w/v) to about 8% (w/v).

Embodiment 12: The composition of embodiment 11, wherein the heparin has a concentration in the composition from about 1% (w/v) to about 4% (w/v).

Embodiment 13: The composition of any one of embodiments 1-12, wherein the EDTA has a concentration in the composition from about 1% (w/v) to about 10% (w/v).

Embodiment 14: The composition of embodiment 13, wherein the EDTA has a concentration in the composition from about 1% (w/v) to about 5% (w/v).

Embodiment 15: The composition of any one of embodiments 1-14, wherein the additional ingredient comprises a thrombolytic agent, and the thrombolytic agent comprises alteplase, streptokinase, reteplase, tenecteplase, urokinase, prourokinase, anistreplase, or a combination thereof.

Embodiment 16: The composition of embodiment 15, wherein the thrombolytic agent comprises alteplase, urokinase, streptokinase, or a combination thereof

Embodiment 17: The composition of embodiment 15, wherein the thrombolytic agent has a concentration in the composition of at least about 0.1% (w/v).

Embodiment 18: The composition of embodiment 17, wherein the thrombolytic agent has a concentration in the composition from about 0.1% (w/v) to about 1.5% (w/v).

Embodiment 19: The composition of any one of embodiments 1-18, wherein the additional ingredient comprises taurolidine, and the taurolidine has a concentration in the composition from about 1% (w/v) to about 8% (w/v).

Embodiment 20: The composition of embodiment 19, wherein the taurolidine has a concentration in the composition from about 1% (w/v) to about 4% (w/v).

Embodiment 21: A sterile composition comprising:
a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and
a thrombolytic agent,
wherein the composition has a pH of at least 6.5 and is biocompatible in a patient's bloodstream.

Embodiment 22: The composition of embodiment 21, wherein the composition has a pH from about 6.5 to about 11.5.

Embodiment 23: The composition of embodiment 22, wherein the composition has a pH from about 6.5 to about 7.5.

Embodiment 24: The composition of embodiment 22, wherein the composition has a pH from about 6.5 to about 10.

Embodiment 25: The composition of embodiment 22, wherein the composition has a pH from about 8.5 to about 11.

Embodiment 26: The composition of any one of embodiments 21-25, further comprising chlorhexidine or a pharmaceutically acceptable salt thereof.

Embodiment 27: The composition of embodiment 26, wherein the chlorhexidine or a pharmaceutically acceptable salt thereof has a concentration in the composition from about 0.5% (w/v) to about 6% (w/v).

Embodiment 28: The composition of embodiment 26, wherein the chlorhexidine or a pharmaceutically acceptable salt thereof has a concentration in the composition from about 0.1 μg/mL to about 100 μg/m L.

Embodiment 29: The composition of any one of embodiments 21-28, further comprising ethanol.

Embodiment 30: The composition of embodiment 29, wherein the ethanol has a concentration in the composition from about 0.1% (w/v) to about 70% (w/v).

Embodiment 31: The composition of any one of embodiments 21-30, wherein the EDTA has a concentration in the composition from about 1% (w/v) to about 10% (w/v).

Embodiment 32: The composition of embodiment 31, wherein the EDTA has a concentration in the composition from about 1% (w/v) to about 5% (w/v).

Embodiment 33: The composition of any one of embodiments 21-32, wherein the thrombolytic agent comprises alteplase, streptokinase, reteplase, tenecteplase, urokinase, prourokinase, anistreplase, or a combination thereof.

Embodiment 34: The composition of embodiment 33, wherein the thrombolytic agent comprises alteplase, urokinase, streptokinase, or a combination thereof

Embodiment 35: The composition of any one of embodiments 21-34, wherein the thrombolytic agent has a concentration in the composition of at least about 0.1% (w/v).

Embodiment 36: The composition of embodiment 35, wherein the thrombolytic agent has a concentration in the composition from about 0.1% (w/v) to about 1.5% (w/v).

Embodiment 37: A sterile composition comprising:
a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and
taurolidine, wherein the taurolidine has a concentration of at least about 0.1% (w/v),
wherein the composition has a pH of at least 6.5 and is biocompatible in a patient's bloodstream.

Embodiment 38: The composition of embodiment 37, wherein the composition has a pH from about 6.5 to about 11.5.

Embodiment 39: The composition of embodiment 38, wherein the composition has a pH from about 6.5 to about 7.5.

Embodiment 40: The composition of embodiment 38, wherein the composition has a pH from about 6.5 to about 10.

Embodiment 41: The composition of embodiment 38, wherein the composition has a pH from about 8.5 to about 11.

Embodiment 42: The composition of any one of embodiments 37-41, further comprising chlorhexidine or a pharmaceutically acceptable salt thereof.

Embodiment 43: The composition of embodiment 42, wherein the chlorhexidine or a pharmaceutically acceptable salt thereof has a concentration in the composition from about 0.5% (w/v) to about 6% (w/v).

Embodiment 44: The composition of embodiment 42, wherein the chlorhexidine or a pharmaceutically acceptable salt thereof has a concentration in the composition from about 0.1 μg/mL to about 100 μg/mL.

Embodiment 45: The composition of any one of embodiments 37-44, further comprising ethanol.

Embodiment 46: The composition of embodiment 45, wherein the ethanol has a concentration in the composition from about 0.1% (w/v) to about 70% (w/v).

Embodiment 47: The composition of any one of embodiments 37-46, wherein the EDTA has a concentration in the composition from about 1% (w/v) to about 10% (w/v).

Embodiment 48: The composition of embodiment 47, wherein the EDTA has a concentration in the composition from about 1% (w/v) to about 5% (w/v).

Embodiment 49: The composition of any one of embodiments 37-48, wherein the taurolidine has a concentration in the composition from about 1% (w/v) to about 8% (w/v).

Embodiment 50: The composition of embodiment 49, wherein the taurolidine has a concentration in the composition from about 1% (w/v) to about 4% (w/v).

Embodiment 51: A sterile composition comprising:
a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and
heparin, wherein the heparin has a concentration of at least about 1% (w/v),
wherein the composition has a pH of at least 6.5 and is biocompatible in a patient's bloodstream.

Embodiment 52: The composition of embodiment 51, wherein the composition has a pH from about 6.5 to about 11.5.

Embodiment 53: The composition of claim 52, wherein the composition has a pH from about 6.5 to about 7.5.

Embodiment 54: The composition of embodiment 52, wherein the composition has a pH from about 6.5 to about 10.

Embodiment 55: The composition of embodiment 52, wherein the composition has a pH from about 8.5 to about 11.

Embodiment 56: The composition of any one of embodiments 51-55, further comprising chlorhexidine or a pharmaceutically acceptable salt thereof.

Embodiment 57: The composition of embodiment 56, wherein the chlorhexidine or a pharmaceutically acceptable salt thereof has a concentration in the composition from about 0.5% (w/v) to about 6% (w/v).

Embodiment 58: The composition of embodiment 56, wherein the chlorhexidine or a pharmaceutically acceptable salt thereof has a concentration in the composition from about 0.1 μg/mL to about 100 μg/m L.

Embodiment 59: The composition of any one of embodiments 51-58, further comprising ethanol.

Embodiment 60: The composition of embodiment 59, wherein the ethanol has a concentration in the composition from about 0.1% (w/v) to about 70% (w/v).

Embodiment 61: The composition of any one of embodiments 51-60, wherein the heparin has a concentration in the composition from about 1% (w/v) to about 8% (w/v).

Embodiment 62: The composition of embodiment 61, wherein the heparin has a concentration in the composition from about 1% (w/v) to about 4% (w/v).

Embodiment 63: The composition of any one of embodiments 51-62, wherein the EDTA has a concentration in the composition from about 1% (w/v) to about 10% (w/v).

Embodiment 64: The composition of embodiment 63, wherein the EDTA has a concentration in the composition from about 1% (w/v) to about 5% (w/v).

### EXAMPLES

### Example 1

A organisms and culture composition included (1) test pathogens of a single isolate from the species Stenotrophomonas maltophilia (ON17), Proteus mirabilis (ON153), Pseudomonas aeruginosa (SK1), and Serratia marcescens (SI<2) as well as (2) two isolates from the species Staphylococcus epidermidis (ON170 and SK9), S. aureus (ON89 and ON184), E. coli (ON29 and SK2) and Candida albicans (ON47 and SK4b).

An antimicrobials composition included a KiteLock™ 4% Sterile Catheter Lock Solution (40 mg/mL tetrasodium EDTA) by SterileCare Inc., which is distinct from standard ‘disodium’ EDTA that is prepared at near-neutral pH; the pH of the KiteLock™ solution is near 11. The high pH does not kill micro-organisms directly but changes EDTA to the tetrasodium form, which has increased microbial killing effects. Chlorhexidine HCl was purchased from Sigma-Aldrich (product #C8527-5G). The antimicrobials composition (1 mg/mL) was made by dissolving the appropriate amount of chlorhexidine HCl powder in distilled water heated to 50° C., allowing the solution to cool and passing it through a 0.22 μm filter.

An assay was made using a minimum inhibitory concentration (MIC) and minimum bactericidal concentration (MBC) and minimum fungicidal concentration (MFC) determination. The MIC was determined by the micro broth dilution method in 96-well plates. Serial two-fold dilutions of tetrasodium EDTA (from 2% to 0.015%), ethanol (from 50% to 0.1%) and chlorhexidine HCl (from 100 μg/mL to 0.025 μg/mL) were prepared in MH broth with a final volume of 90 μL per well. A 10 μL containing 1×10⁵ bacterial cells or 2×10³ fungal cells were added to each well. The inoculated plates were covered with a lid, sealed with Parafilm, and incubated for 24 h at 37° C. with slight rocking on a tilting platform shaker. After incubation, the optical density at 600 nm (OD 600) of the cultures in each well was measured using an xMark™ Microplate Absorbance Spectrophotometer (Bio-Rad). The MIC was defined as the lowest concentration of antimicrobial compound at which the culture OD₆₀₀ values were similar to uninoculated control wells. MBCs and MFCs were determined by transferring 100 μL from each well with no apparent growth onto appropriate agar plates, followed by incubation for 24 h at 37° C.

A fourth composition and solution included tetrasodium EDTA with ethanol or chlorhexidine HCl. This fourth composition and solution were created using checkerboard titration methods using micro broth dilution in 96-well microtiter plates. The concentrations of antimicrobials used were based on previously determined MIC values. Briefly, 200 μL of two-fold dilutions of tetrasodium EDTA and ethanol or chlorhexidine HCl were prepared in MH or MH II broth with standardized cell suspension. The plate contained decreasing concentrations of tetrasodium EDTA (2%-0.015%) in columns 1-10 and decreasing concentrations of ethanol (50%-0.4%) or chlorhexidine HCl (50 μg/mL-0.0125 μg/mL) in rows A-H. Then, 10 μL of standardized cell suspension was added to each well. Microtiter plates were incubated at 37° C. for 24 h, and the results were analyzed. Each test was performed in duplicate and included a growth control without adding any antimicrobials.

A biofilm cultivation cell composition was provided. This fifth composition was created using an MBEC Assay® biofilm inoculator, consisting of a polystyrene lid with 96 downward-protruding pegs and a corresponding base used to grow biofilms. A standardized inoculum was diluted in an appropriate biofilm growth medium to achieve a viable cell count of 1.5×10⁶ CFU/mL of bacterial cells or 5×10⁵ CFU/mL of fungal cells. Then, 150 μL of this inoculum was transferred into each appropriate well, and the peg lids were inserted into the microtiter plates. The plates were sealed with Parafilm and were incubated at optimum temperature for 48 h with slight rocking for bacteria and shaking at 200 rpm for fungal strains. After incubation, the peg lid was removed from the base and rinsed twice with sterile phosphate-buffered saline (PBS) for 2 min to remove loosely attached non-sessile cells. Before the antimicrobial challenge, the pegs in column 1 (n=8) were considered the biofilm growth control; these pegs were removed from the lids, placed into 200 μL of recovery medium, and analyzed for starting biofilm cell numbers as described below. The rinsed pegs were placed into new 96-well plates containing two-fold dilutions of antimicrobials such as tetrasodium EDTA (4%-0.0125%), ethanol (100%-0.2%), and chlorhexidine HCl (100 μg/mL-0.4 μg/mL) in 200 μL of suitable biofilm growth medium per well and incubated at optimum temperature for 24 h. After the antimicrobial challenge, the pegs were rinsed twice with sterile PBS for 2 min and placed into a new 96-well plate containing 200 μL of recovery medium. The recovery plates were sealed with Parafilm, and biofilm cells were dislodged from the pegs by sonication for 30 min with a Branson 3510 bath sonicator. The biofilm cells in the recovery medium were serially diluted, and a drop dilution assay was performed to enumerate the viable cells. MBEC values were determined as the minimum concentration of antimicrobials that yielded a viable cell count at or lower than the 125 CFU/mL detection limit.

Determining the fractional biofilm eradication concentration (FBEC) index included the steps of (1) identifying synergistic antimicrobial effects of tetrasodium EDTA with either ethanol or chlorhexidine HCl on established biofilms, (2) using the ‘checkerboard dilution method’ where (3) pegs containing biofilms were treated with a combination of tetrasodium EDTA and ethanol or with tetrasodium EDTA and chlorhexidine HCl in 200 μL of two-fold dilutions inappropriate biofilm growth medium. This was followed by step (4) that included eight dilution steps of tetrasodium EDTA (4%-0.015%) either with ethanol (50%-0.4%) or chlorhexidine HCl (50 μg/mL-0.4 μg/mL) and where eight growth controls are analyzed for synergistic biofilm eradication. In step (4), microtiter plates are incubated at 37° C. for 24 h. then (6), after incubation, the bacterial and fungal cells were dislodged from the pegs into the recovery medium described above.

Three 10-μL aliquots, for a total of 30 μL from each well of recovery medium, were spotted on MH agar plates and incubated for 24 h at 37° C. The FBEC is the minimum concentration of antimicrobials in combination that completely inhibited bacterial or fungal growth on agar plates. The FBEC determination is a modification of the FICI.

Determining rapid biofilm eradication by tetrasodium EDTA, ethanol, and chlorhexidine HCl alone and in combination was performed. After biofilm formation, control pegs (n=6) were removed and analyzed to determine the starting biofilm cell numbers via the drop dilution method. The 48-h old biofilms on the pegs were exposed to different concentrations of test antimicrobials, dissolved in an appropriate growth medium, for two h to evaluate their efficacy alone and in combination. Antimicrobial solutions tested against each organism included each agent alone at the MBEC, double combinations at the FBEC, and triple combinations ranging from 5 to 20% ethanol, 2.5-5 μg/mL chlorhexidine HCl and 1-3% tetrasodium EDTA. Following treatment, pegs were washed twice with sterile PBS, and the biofilm cells were dislodged into recovery medium and enumerated as described above.

Antimicrobial activity of tetrasodium EDTA alone and in combination with either ethanol or chlorhexidine HCl against planktonic cells was determined. All three antimicrobials significantly inhibited the growth of all test organisms with MICs ranging from 0.063% to 2% for tetrasodium EDTA, 3.125%-12.5% for ethanol, and 0.1 μg/mL-50 μg/mL for chlorhexidine HCl. Synergy (FICI<0.5) was detected with the combination of tetrasodium EDTA with ethanol for all test Gram-positive and fungal strains, whereas partial synergy (0.5<FICI<1.0) was observed for all Gram-negative strains. The combination of tetrasodium EDTA with chlorhexidine HCl showed indifferent activity (1<FICI<4) against 4 of 12 test strains and synergistic or partially synergistic activity against the eight remaining strains (Tables 1A-1C below).

TABLE 1A
Minimum Inhibitory Concentration of Tetrasodium
EDTA, Ethanol, and Chlorhexidine HCl alone.

MIC

| | EDTA | Ethanol | Chlorhexidine HCl |
|---|---|---|---|
| Organism | (% w/v) | (% w/v) | (μg/mL) |
| Staphylococcus | 0.063 | 3.125 | 0.1 |
| epidermidis ON170 | | | |
| S. epidermidis SK9 | 0.063 | 6.25 | 0.1 |
| Staphylococcus | 0.063 | 6.25 | 0.1 |
| aureus ON89 | | | |
| MRSA ON184 | 0.063 | 6.25 | 0.2 |
| Stenotrophomonas | 0.063 | 6.25 | 0.8 |
| maltophilia ON17 | | | |
| Pseudomonas | 0.25 | 3.125 | 1.6 |
| aeruginosa SK1 | | | |
| Serratia | 2 | 6.25 | 12.5 |
| marcescens SK2 | | | |
| Proteus mirabilis | 1 | 6.25 | 50 |
| ON153 | | | |
| Escherichia coli | 0.5 | 12.5 | 0.4 |
| ON29 | | | |
| E. coli SK2 | 0.5 | 12.5 | 0.4 |
| Candida albicans | 1 | 6.25 | 1.6 |
| SK4b | | | |
| C. albicans ON47 | 1 | 6.25 | 1.6 |

TABLE 1B
Fractional Inhibitory Concentration Index (FICI)
of Tetrasodium EDTA in Combination with Ethanol

| Organism | EDTA (% w/v) | Ethanol (% w/v) | FICI | |
|---|---|---|---|---|
| Staphylococcus | 0.015 | 0.4 | 0.36 | (S) |
| epidermidis ON170 | | | | |
| S. epidermidis SK9 | 0.015 | 0.4 | 0.3 | (S) |
| Staphylococcus | 0.015 | 0.4 | 0.3 | (S) |
| aureus ON89 | | | | |
| MRSA ON184 | 0.015 | 0.4 | 0.3 | (S) |
| Stenotrophomonas | 0.031 | 0.4 | 0.72 | (PS) |
| maltophilia ON17 | | | | |
| Pseudomonas | 0.031 | 3.125 | 0.56 | (PS) |
| aeruginosa SK1 | | | | |
| Serratia | 0.5 | 3.125 | 0.75 | (PS) |
| marcescens SK2 | | | | |
| Proteus mirabilis | 0.5 | 1.6 | 0.75 | (PS) |
| ON153 | | | | |
| Escherichia coli | 0.25 | 3.125 | 0.75 | (PS) |
| ON29 | | | | |
| E. coli SK2 | 0.015 | 6.25 | 0.53 | (PS) |
| Candida albicans | 0.25 | 1.6 | 0.5 | (S) |
| SK4b | | | | |
| C. albicans ON47 | 0.25 | 0.4 | 0.314 | (S) |

TABLE 1C
Fractional Inhibitory Concentration Index (FICI) of Tetrasodium
EDTA in Combination with Chlorhexidine HCl

| | | Chlorhexidine HCl | | |
|---|---|---|---|---|
| Organism | EDTA (% w/v) | (μg/mL) | FICI | |
| Staphylococcus | 0.063 | 0.05 | 1.5 | (I) |
| epidermidis ON170 | | | | |
| S. epidermidis SK9 | 0.015 | 0.025 | 0.5 | (S) |
| Staphylococcus | 0.008 | 0.1 | 1.126 | (I) |
| aureus ON89 | | | | |
| MRSA ON184 | 0.008 | 0.2 | 1.126 | (I) |
| Stenotrophomonas | 0.015 | 0.2 | 0.5 | (S) |
| maltophilia ON17 | | | | |
| Pseudomonas | 0.5 | 0.8 | 2.5 | (I) |
| aeruginosa SK1 | | | | |
| Serratia | 0.063 | 1.5 | 0.16 | (S) |
| marcescens SK2 | | | | |
| Proteus mirabilis | 0.063 | 0.8 | 0.08 | (S) |
| ON153 | | | | |
| Escherichia coli | 0.008 | 0.2 | 0.516 | (PS) |
| ON29 | | | | |
| E. coli SK2 | 0.008 | 0.1 | 0.266 | (S) |
| Candida albicans | 0.063 | 0.8 | 0.596 | (PS) |
| SK4b | | | | |
| C. albicans ON47 | 0.125 | 0.8 | 0.658 | (PS) |

The three antimicrobial agents displayed broad-spectrum microbicidal activity against the 12 test organisms. MBC or MFC values of all test antimicrobials were equal to or higher than their respective MICs. The combination of tetrasodium EDTA with either ethanol or chlorhexidine HCl showed synergistic and partially synergistic activity against all the test strains except S. epidermidis ON170, which showed additive activity with an FMCI of 1.0. The nature of interaction found in FICI was not always the same as the FMCI. However, none of the tested tetrasodium EDTA, ethanol, or chlorhexidine HCl combinations showed antagonism concerning the FICI and FMCI values. These results are shown in Tables 2A-2C.

TABLE 2A
Minimum Bactericidal Concentration (MBC) or Minimum
Fungicidal Concentration (MFC) of Tetrasodium
EDTA, Ethanol, and Chlorhexidine HCl alone.

MBC/MFC

| | EDTA | Ethanol | Chlorhexidine HCl |
|---|---|---|---|
| Organism | (% w/v) | (% w/v) | (μg/mL) |
| Staphylococcus | 0.5 | 6.25 | 0.8 |
| epidermidis ON170 | | | |
| S. epidermidis SK9 | 0.5 | 12.5 | 0.8 |
| Staphylococcus | 1 | 25 | 0.8 |
| aureus ON89 | | | |
| MRSA ON184 | 2 | 25 | 1.6 |
| Stenotrophomonas | 1 | 6.25 | 3.125 |
| maltophilia ON17 | | | |
| Pseudomonas | 1 | 12.5 | 3.125 |
| aeruginosa SK1 | | | |
| Serratia | 2 | 12.5 | 25 |
| marcescens SK2 | | | |
| Proteus mirabilis | 2 | 12.5 | 50 |
| ON153 | | | |
| Escherichia coli | 1 | 25 | 0.8 |
| ON29 | | | |
| E. coli SK2 | 0.5 | 25 | 0.8 |
| Candida albicans | 1 | 6.25 | 3.125 |
| SK4b | | | |
| C. albicans ON47 | 1 | 6.25 | 3.125 |

TABLE 2B
Fractional Microbicidal Concentration Index (FMCI)
of Tetrasodium EDTA in Combination with Ethanol

| Organism | EDTA (% w/v) | Ethanol (% w/v) | FMCI | |
|---|---|---|---|---|
| Staphylococcus | 0.031 | 1.6 | 0.318 | (S) |
| epidermidis ON170 | | | | |
| S. epidermidis SK9 | 0.031 | 3.125 | 0.312 | (S) |
| Staphylococcus | 0.25 | 3.125 | 0.375 | (S) |
| aureus ON89 | | | | |
| MRSA ON184 | 0.031 | 3.125 | 0.14 | (S) |
| Stenotrophomonas | 0.125 | 1.6 | 0.381 | (S) |
| maltophilia ON17 | | | | |
| Pseudomonas | 0.031 | 6.25 | 0.531 | (PS) |
| aeruginosa SK1 | | | | |
| Serratia | 0.25 | 6.25 | 0.625 | (PS) |
| marcescens SK2 | | | | |
| Proteus mirabilis | 0.063 | 6.25 | 0.53 | (PS) |
| ON153 | | | | |
| Escherichia coli | 0.25 | 6.25 | 0.5 | (S) |
| ON29 | | | | |
| E. coli SK2 | 0.25 | 6.25 | 0.75 | (PS) |
| Candida albicans | 0.25 | 3.125 | 0.75 | (PS) |
| SK4b | | | | |
| C. albicans ON47 | 0.25 | 3.125 | 0.625 | (PS) |

TABLE 2C
Fractional Microbicidal Concentration Index (FMCI) of
Tetrasodium EDTA in Combination with Chlorhexidine HCl

| | | Chlorhexidine HCl | | |
|---|---|---|---|---|
| Organism | EDTA (% w/v) | (μg/mL) | FMCI | |
| Staphylococcus | 0.25 | 0.4 | 1 | (A) |
| epidermidis ON170 | | | | |
| S. epidermidis SK9 | 0.008 | 0.4 | 0.516 | (PS) |
| Staphylococcus | 0.008 | 0.2 | 0.375 | (S) |
| aureus ON89 | | | | |
| MRSA ON184 | 0.125 | 0.4 | 0.312 | (S) |
| Stenotrophomonas | 0.031 | 0.4 | 0.16 | (S) |
| maltophilia ON17 | | | | |
| Pseudomonas | 0.031 | 1.6 | 0.543 | (PS) |
| aeruginosa SK1 | | | | |
| Serratia | 0.125 | 1.6 | 0.126 | (S) |
| marcescens SK2 | | | | |
| Proteus mirabilis | 0.125 | 1.6 | 0.09 | (S) |
| ON153 | | | | |
| Escherichia coli | 0.008 | 0.2 | 0.258 | (S) |
| ON29 | | | | |
| E. coli SK2 | 0.015 | 0.2 | 0.258 | (S) |
| Candida albicans | 0.125 | 0.8 | 0.381 | (S) |
| SK4b | | | | |
| C. albicans ON47 | 0.031 | 1.6 | 0.51 | (PS) |

Compositions of tetrasodium EDTA alone and in combination with either ethanol or chlorhexidine HCl against 48-h old, preformed biofilms may be created using a single antimicrobial agent, effective at eradicating preformed biofilms of test pathogens, with concentrations between 4% to 0.0125% of tetrasodium EDTA, 100%-0.2% of ethanol, and 100 μg/mL-0.8 μg/mL of chlorhexidine HCl. As per CLSI guidelines, the MBEC is defined as the minimum concentration of an antimicrobial that eradicates 99.9% of micro-organisms (i.e., 3-log reduction) in a biofilm state compared with their respective growth controls in similar conditions. All antimicrobials achieved >99.99% (i.e., 4-log reduction) killing of bacterial biofilm cells, whereas the starting biofilm cell numbers for C. albicans were not enough to achieve a clinically recommended standard of biofilm killing.

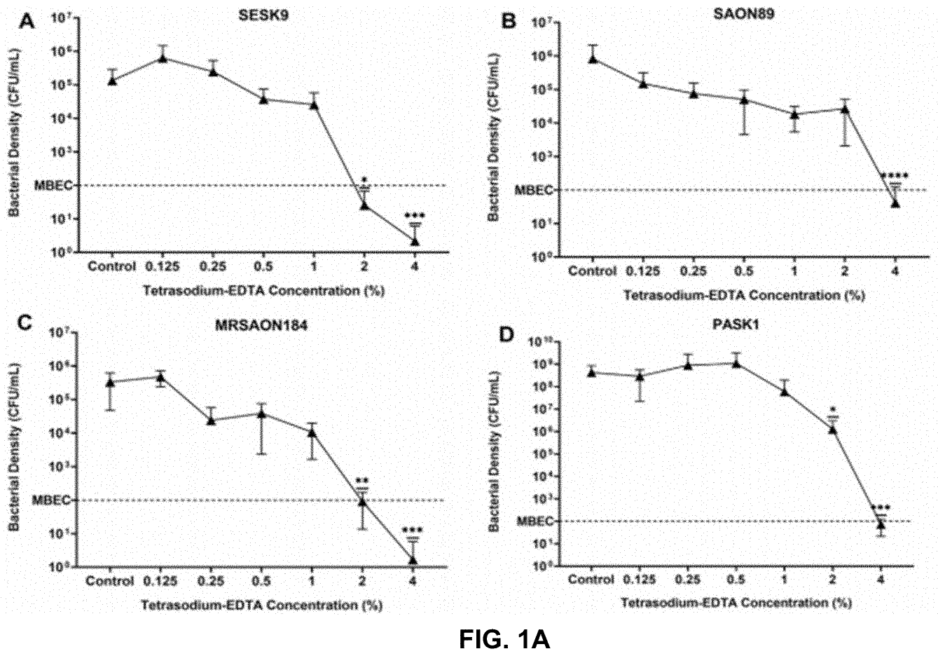

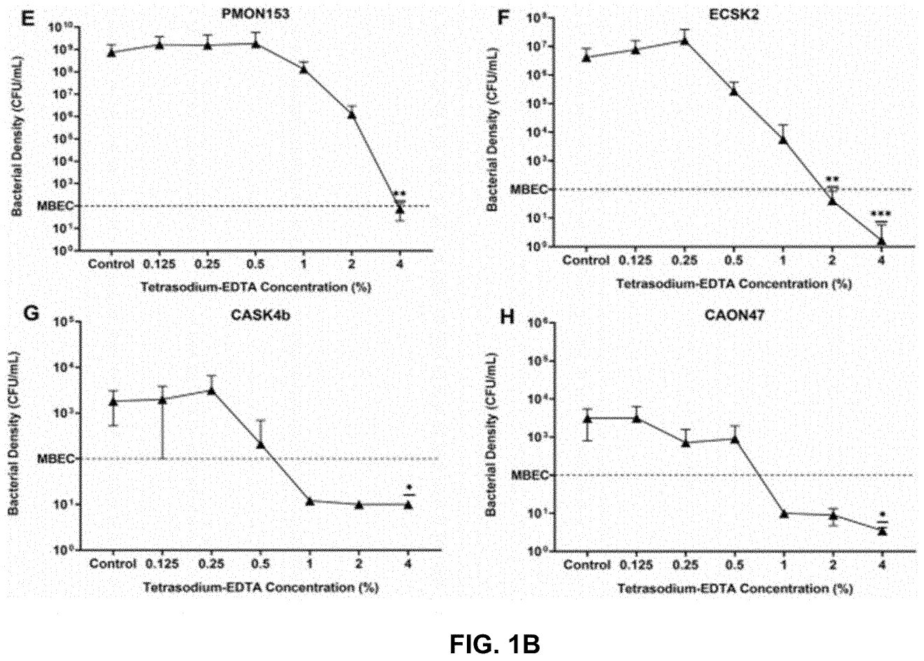

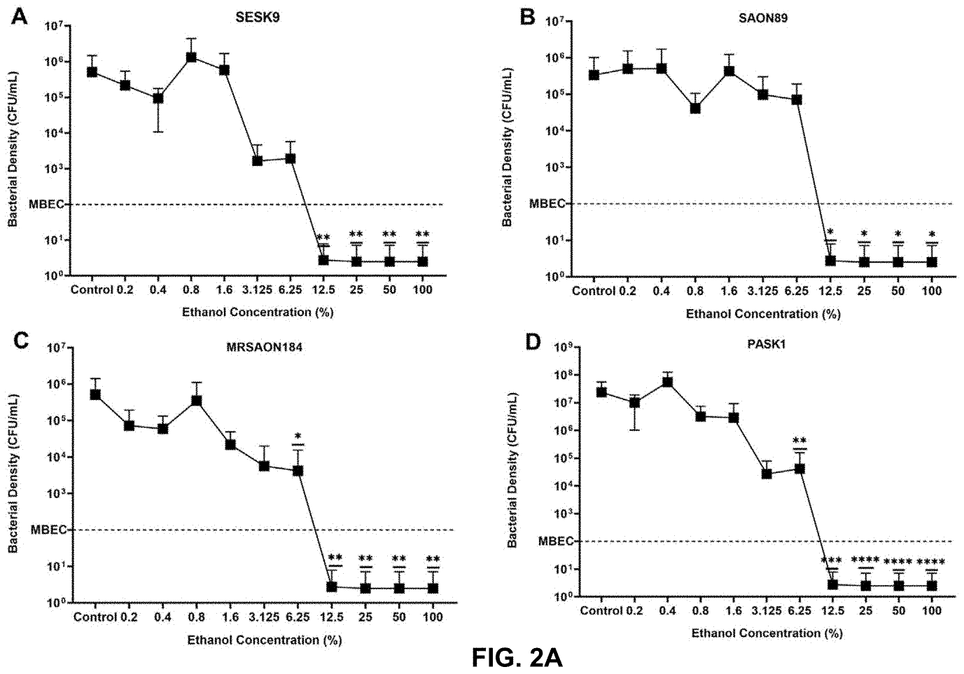

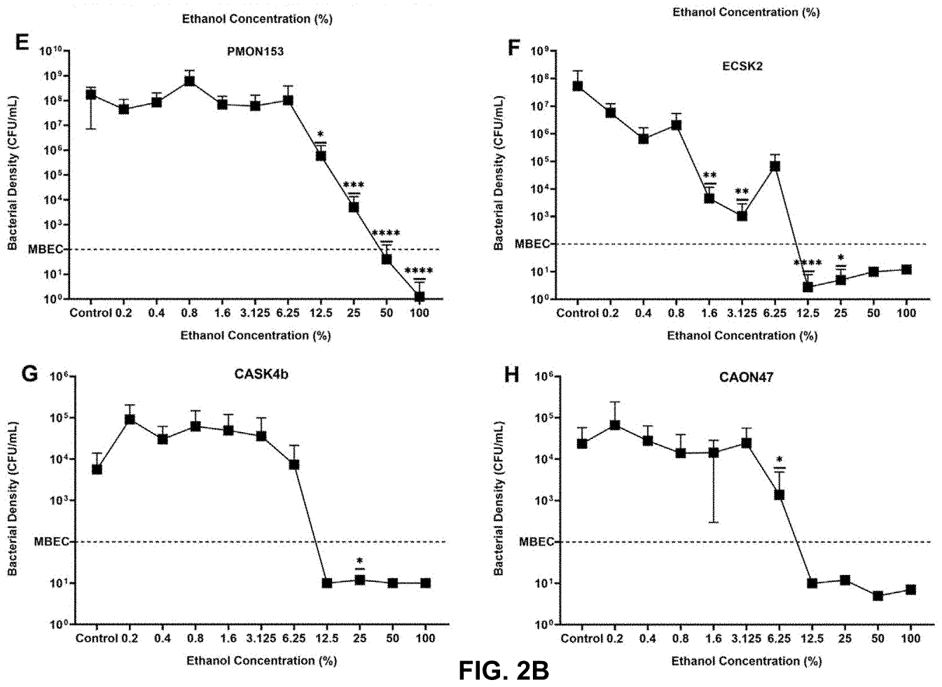

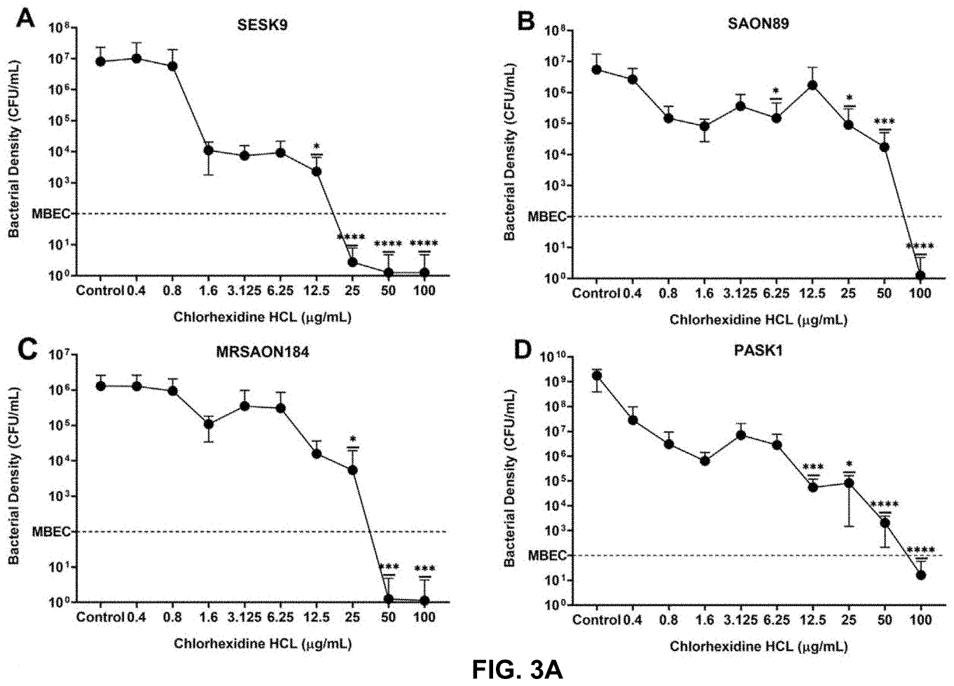

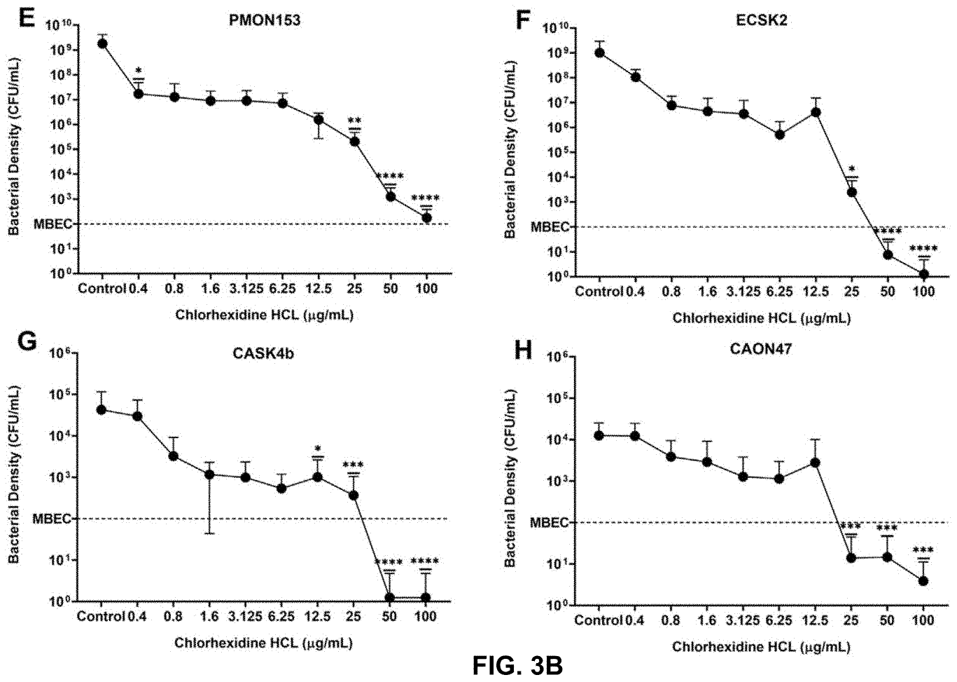

The MBEC of each antimicrobial agent against each test strain was established, and the data were plotted as the log reduction in the number of CFU (FIGS. 1A-3B). When tetrasodium EDTA was combined with either ethanol or chlorhexidine HCl, they exhibited a synergistic effect against all test strains in the study (Table 3). According to the FBEC index, the concentration of tetrasodium EDTA in combination was decreased from 1/8- to 1/64-fold (with ethanol) and 1/16- to 1/64-fold (with chlorhexidine) in comparison with its original MBEC values. Also, the required concentrations dropped by 1/4- to 1/16-fold for ethanol and 1/8- to 1/32-fold for chlorhexidine HCl when combined with tetrasodium EDTA (Tables 3A-3C).

TABLE 3A
Minimum Biofilm Eradication Concentration (MBEC) of Tetrasodium
EDTA in Combination with Chlorhexidine HCl

| | MBEC | | |
|---|---|---|---|
| | EDTA | Ethanol | Chlorhexidine HCl |
| Organism | (% w/v) | (% w/v) | (μg/mL) |
| Staphylococcus | 2 | 12.5 | 25 |
| epidermidis SK9 | | | |
| Staphylococcus | 4 | 12.5 | 100 |
| aureus ON89 | | | |
| MRSA ON184 | 2 | 12.5 | 50 |
| Pseudomonas | 4 | 12.5 | 100 |
| aeruginosa SK1 | | | |
| Proteus mirabilis | 4 | 50 | 100 |
| ON153 | | | |
| Escherichia coli | 2 | 12.5 | 50 |
| SK2 | | | |
| Candida albicans | 1 | 12.5 | 50 |
| SK4b | | | |
| C. albicans ON47 | 1 | 12.5 | 25 |

TABLE 3B
Fractional Biofilm Eradication Concentration Index
(FBECI) of Tetrasodium EDTA Combined with Ethanol

| Organism | EDTA (% w/v) | Ethanol (% w/v) | FBECI |
|---|---|---|---|
| Staphylococcus | 0.031 | 1.6 | 0.14 (S) |
| epidermidis SK9 | | | |
| Staphylococcus | 0.063 | 0.8 | 0.08 (S) |
| aureus ON89 | | | |
| MRSA ON184 | 0.063 | 1.6 | 0.16 (S) |
| Pseudomonas | 0.125 | 3.125 | 0.28 (S) |
| aeruginosa SK1 | | | |
| Proteus mirabilis | 0.125 | 6.25 | 0.16 (S) |
| ON153 | | | |
| Escherichia coli | 0.125 | 1.6 | 0.19 (S) |
| SK2 | | | |
| Candida albicans | 0.125 | 1.6 | 0.253 (S) |
| SK4b | | | |
| C. albicans ON47 | 0.125 | 1.6 | 0.253 (S) |

TABLE 3B
Fractional Biofilm Eradication Concentration Index (FBECI)
of Tetrasodium EDTA Combined with Cholorhexidine HCl

| | | Chlorhexidine HCl | |
|---|---|---|---|
| Organism | EDTA (% w/v) | (μg/mL) | FBECI |
| Staphylococcus | 0.125 | 3.125 | 0.19 (S) |
| epidermidis SK9 | | | |
| Staphylococcus | 0.125 | 3.125 | 0.06 (S) |
| aureus ON89 | | | |
| MRSA ON184 | 0.031 | 6.25 | 0.14 (S) |
| Pseudomonas | 0.25 | 3.125 | 0.093 (S) |
| aeruginosa SK1 | | | |
| Proteus mirabilis | 0.125 | 3.125 | 0.06 (S) |
| ON153 | | | |
| Escherichia coli | 0.031 | 3.125 | 0.08 (S) |
| SK2 | | | |
| Candida albicans | 0.063 | 6.25 | 0.18 (S) |
| SK4b | | | |
| C. albicans ON47 | 0.031 | 6.25 | 0.28 (S) |

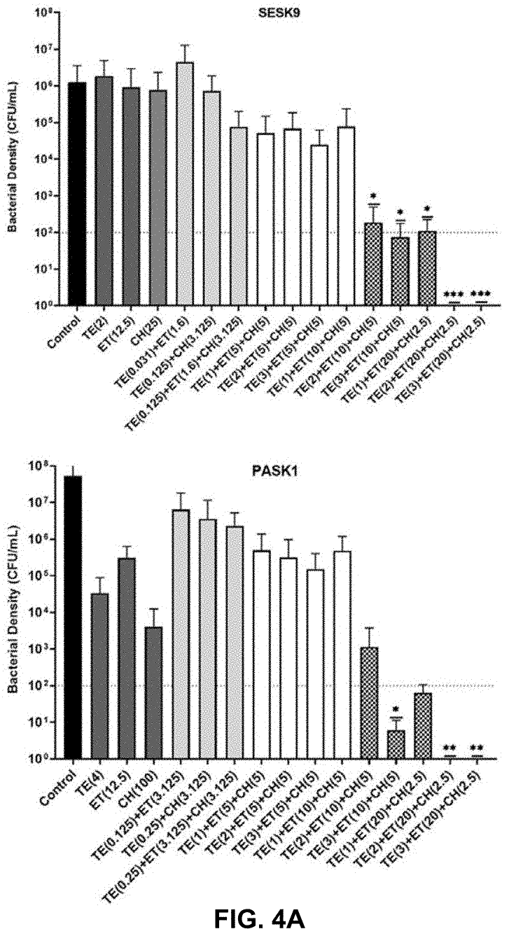

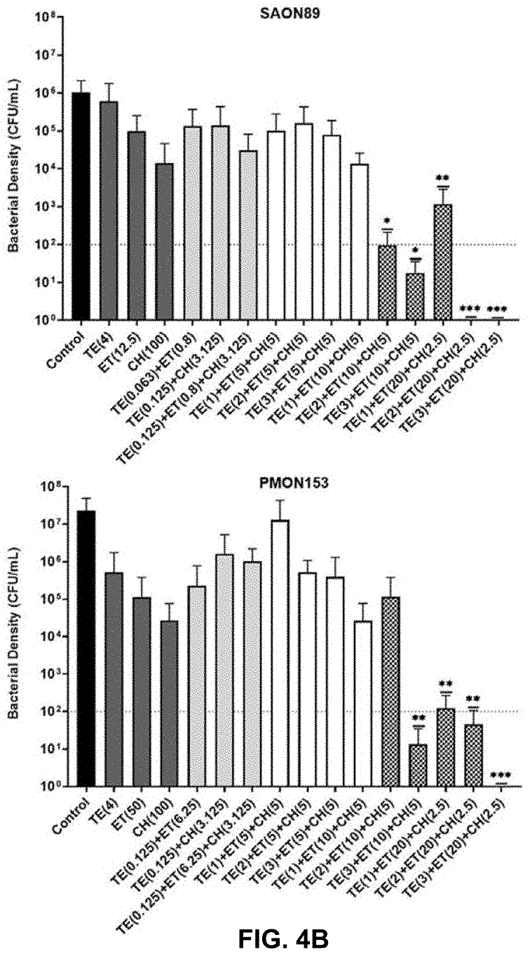

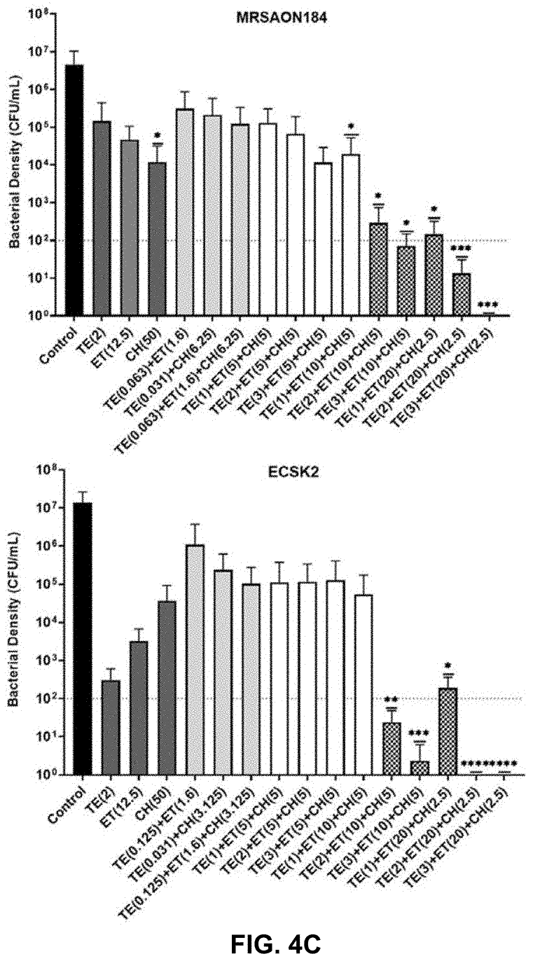

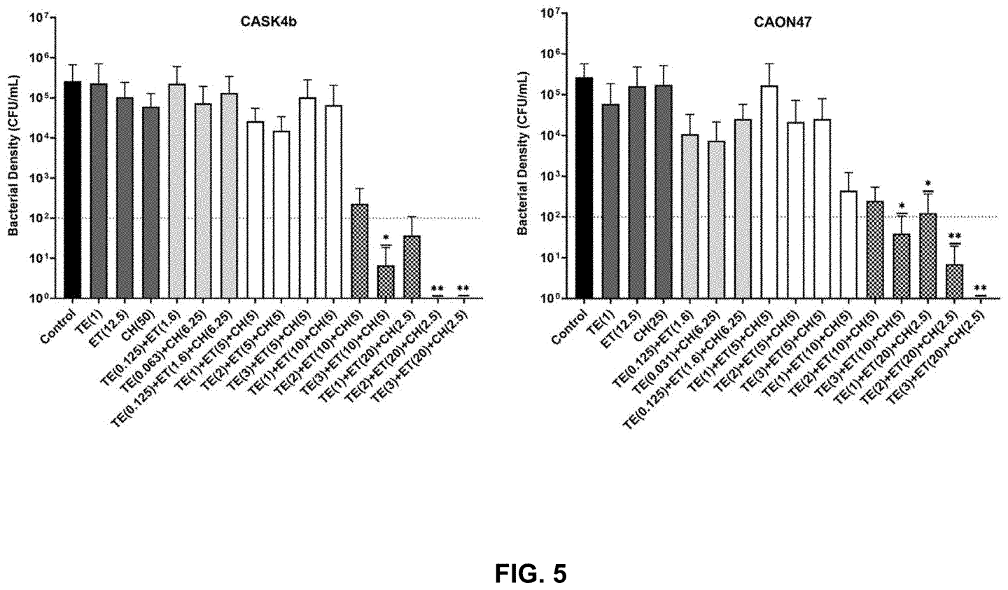

The method for rapid biofilm eradication ability of test antimicrobials alone and in combination against 48-h-old biofilms within two h exposure time included (1) choosing different concentrations of test antimicrobials to assess their potency in eradicating preformed biofilms of study organisms within two h. Then (2) the quantitative recovery from biofilms following exposure to the antimicrobial solutions for bacterial strains (FIGS. 4A-4C) and fungal strains (FIG. 5) was evaluated. Then (3) the exposure to the MBEC of tetrasodium EDTA, ethanol, or chlorhexidine HCl alone was measured as well as in several double and triple combinations of FBEC of antimicrobials failed to eradicate the preformed biofilms after two h of exposure. Then (4) all tested bacterial and fungal biofilms were entirely eradicated by the triple combination of 20% ethanol and 2.5 μg/mL chlorhexidine HCl in 3% tetrasodium EDTA (FIGS. 4A-5).

A triple combination of 20% ethanol and 2.5 μg/mL chlorhexidine HCl in 2% tetrasodium EDTA ultimately killed all biofilm cells except for three strains (MRSA ON184, P. mirabilis ON153, and C. albicans SK4b), but even for these strains, the viable cells were significantly reduced to at or near the limit of detection. Likewise, a combination of 1% tetrasodium EDTA with 20% ethanol and 2.5 μg/mL chlorhexidine HCl significantly reduced the viable cells in six of eight test organisms in comparison with their respective controls. A triple combination of 3% tetrasodium EDTA with 10% ethanol and five μg/mL chlorhexidine HCl also showed a significant reduction in viable biofilm cells of all test organisms within the 2-h contact time.

The results demonstrated that all test antimicrobials had efficient antimicrobial activity against planktonic and biofilm cells of test bacterial and fungal strains when exposed for 24 h. The combination of tetrasodium EDTA and ethanol was synergistic against planktonic cells of 6 of 12 strains tested, as measured by inhibition (FICI) and microbicidal (FMCI) activity. The interactions between tetrasodium EDTA and chlorhexidine HCl were categorized into synergistic, partially synergistic, additive, and indifferent activity against the test bacterial and fungal strains. It is noteworthy that there was no evidence of antagonistic activity between the three agents against planktonic cells in any tested combinations. We also tested the biofilm eradication ability of test antimicrobials against 48-h-old biofilms of bacterial and fungal strains within a 24-h exposure; 4% tetrasodium EDTA, 5% ethanol, and 100 μg/mL chlorhexidine HCl alone were able to eradicate all established biofilms following 24 h of treatment. As expected, biofilm cells were more resistant for each organism than planktonic cells. When tetrasodium EDTA was combined with ethanol or chlorhexidine HCl and used to treat biofilms, the agents worked synergistically, showing a remarkable reduction in concentrations compared with the MBEC values of single test antimicrobials. In many cases, the concentration of each agent required was near or lower than the MICs measured against planktonic cells. This strongly indicated that these three antimicrobials could be successfully used together to kill pathogenic microbes.

The combinations of antimicrobial agents showed efficient microbicidal activity against organisms within a reasonable contact time. Based on the results obtained from previous studies and the present study, concentrations of all three agents were chosen to optimize the effective combinations to eradicate biofilms within a selected 2-h exposure. The present study demonstrated that triple combinations of either 3% tetrasodium EDTA with 10% ethanol and 5 μg/mL chlorhexidine HCl or of 3% tetrasodium EDTA with 20% ethanol and 2.5 μg/mL chlorhexidine HCl completely eradicated 48-h-old biofilms of all of the test organisms following a 2-h exposure. In comparison with their individual antimicrobial effects, the combination of test antimicrobials significantly decreased the viable cells both of bacterial and fungal biofilms. The decrease in the ethanol concentration was compensated with an increased concentration of tetrasodium EDTA, and the effect was further accelerated with the addition of chlorhexidine HCl. The reduced ethanol concentration in the present study sets a more significant margin of safety from adverse reactions. In addition to improving safety, combination therapy may also decrease the risk of antimicrobial resistance among pathogens by reducing selection pressure. In addition, chlorhexidine concentrations above 2% have fewer human erythrocytes and neutrophils in vitro.

Additionally, toxicity of chlorhexidine is directly proportional to its concentration used. Considering this fact, the concentration of chlorhexidine HCl used in the triple combination was 0.00025% (w/v) in the present study.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present systems and methods, which, as a matter of language, might be said to fall therebetween.

What is claimed is:

1. A sterile composition comprising:
a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and
an additional ingredient selected from the group consisting of heparin, taurolidine, a thrombolytic agent, or a combination thereof,
wherein the composition has a pH of at least 6.5 and is biocompatible in a patient's bloodstream.

2. The composition of claim 1, wherein the composition has a pH from about 6.5 to about 11.5.

3. The composition of claim 2, wherein the composition has a pH from about 6.5 to about 7.5.

4. The composition of claim 2, wherein the composition has a pH from about 6.5 to about 10.

5. The composition of claim 2, wherein the composition has a pH from about 8.5 to about 11.

6. The composition of any one of claims 1-5, further comprising chlorhexidine or a pharmaceutically acceptable salt thereof.

7. The composition of claim 6, wherein the chlorhexidine or a pharmaceutically acceptable salt thereof has a concentration in the composition from about 0.5% (w/v) to about 6% (w/v).

8. The composition of claim 6, wherein the chlorhexidine or a pharmaceutically acceptable salt thereof has a concentration in the composition from about 0.1 μg/mL to about 100 μg/m L.

9. The composition of any one of claims 1-8, further comprising ethanol.

10. The composition of claim 9, wherein the ethanol has a concentration in the composition from about 0.1% (w/v) to about 70% (w/v).

11. The composition of any one of claims 1-10, wherein the additional ingredient comprises heparin, and the heparin has a concentration in the composition from about 1% (w/v) to about 8% (w/v).

12. The composition of claim 11, wherein the heparin has a concentration in the composition from about 1% (w/v) to about 4% (w/v).

13. The composition of any one of claims 1-12, wherein the EDTA has a concentration in the composition from about 1% (w/v) to about 10% (w/v).

14. The composition of claim 13, wherein the EDTA has a concentration in the composition from about 1% (w/v) to about 5% (w/v).

15. The composition of any one of claims 1-14, wherein the additional ingredient comprises a thrombolytic agent, and the thrombolytic agent comprises alteplase, streptokinase, reteplase, tenecteplase, urokinase, prourokinase, anistreplase, or a combination thereof.

16. The composition of claim 15, wherein the thrombolytic agent comprises alteplase, urokinase, streptokinase, or a combination thereof

17. The composition of claim 15, wherein the thrombolytic agent has a concentration in the composition of at least about 0.1% (w/v).

18. The composition of claim 17, wherein the thrombolytic agent has a concentration in the composition from about 0.1% (w/v) to about 1.5% (w/v).

19. The composition of any one of claims 1-18, wherein the additional ingredient comprises taurolidine, and the taurolidine has a concentration in the composition from about 1% (w/v) to about 8% (w/v).

20. The composition of claim 19, wherein the taurolidine has a concentration in the composition from about 1% (w/v) to about 4% (w/v).

21. A sterile composition comprising:
a salt of ethylene diamine tetraacetic acid (EDTA) in solution, wherein the salt of EDTA comprises tri-sodium or tetra-sodium EDTA, wherein the EDTA has a concentration in the composition from about 1% (w/v) to about 15% (w/v); and
a thrombolytic agent,
wherein the composition has a pH of at least 6.5 and is biocompatible in a patient's bloodstream.
